(19) United States
(12) Patent Application Publication
(10) Pub. No.: US 2014/0099634 A1
(43) Pub. Date: Apr. 10, 2014

(54) USE OF PRE-MRNA SPLICING IN PLATELET CELLS FOR THE DIAGNOSIS OF DISEASE

(71) Applicants: Andrew S. WEYRICH, Salt Lake City, UT (US); Hansjorg SCHWERTZ, Salt Lake City, UT (US); Guy A. ZIMMERMAN, Salt Lake City, UT (US)

(72) Inventors: Andrew S. WEYRICH, Salt Lake City, UT (US); Hansjorg SCHWERTZ, Salt Lake City, UT (US); Guy A. ZIMMERMAN, Salt Lake City, UT (US)

(21) Appl. No.: 13/693,937

(22) Filed: Dec. 4, 2012

Related U.S. Application Data
(63) Continuation of application No. 12/452,333, filed on Jul. 30, 2010, now abandoned.
(60) Provisional application No. 60/936,593, filed on Jun. 20, 2007.
(60) Provisional application No. 60/936,528, filed on Jun. 20, 2007.

(51) Int. Cl. C12Q 1/68

(52) U.S. Cl. CPC C12Q 1/6881
USPC 435/6.11; 435/6.12

(57) ABSTRACT

The invention relates to materials and procedures for identifying or using tissue factor (TF) pre-mRNA splicing, Clk 1 activity or TF-dependent coagulation in platelet cells for the diagnosis, prognosis, or prediction of a disease or disorder associated with disordered coagulation. Since activated platelets splice pre-mRNAs to generate inflammatory and thrombotic mediators that contribute to diseases such as sepsis and septic shock, (TF) pre-mRNA splicing in platelets is an indicator of inflammatory and thrombotic disease states. TF pre-mRNA splicing in platelets is correlated with sepsis, increased age (≧65), APACHE II score, and bacteremia. Thus, TF snRNA expression patterns in platelets may be used for the diagnosis, prognosis, or prediction of a disease or disorder associated with disordered coagulation, for example, patients that are at a higher risk for severe sepsis, organ failure, and death.

### CROSS-REFERENCE TO RELATED APPLICATIONS

This application is a continuation of U.S. patent application Ser. No. 12/452,333, filed Jul. 30, 2010, which is the U.S. National Stage of PCT International Application Number PCT/US2008/007755, filed Jun. 20, 2008, which claims the benefit of U.S. Provisional Patent Application No. 60/936,528, filed Jun. 20, 2007, which is related to U.S. Provisional Application No. 60/936,593, filed Jun. 20, 2007. The contents of the foregoing applications are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Apr. 16, 2013, is named 0324SEQUENCE_LISTING.txt and is 1,799 bytes in size.

### TECHNICAL FIELD

The invention relates to biotechnology generally and more particularly to the use of Tissue Factor (TF) pre-mRNA splicing, TF-dependent coagulation and/or stabilization of a platelet thrombus to provide a diagnosis, prognosis, and/or prediction for a coagulation related disorder, disease or condition.

### BACKGROUND

The following discussion of the background of the invention is provided to aid the reader in understanding the invention and is not an admission that anything herein describes or constitutes prior art.

Because platelets lack nuclei, it was presumed that their transcriptome is fixed and simply reflects their megakaryocyte-derived mRNA portfolio²⁵. However, Post-transcriptional signaling pathways are used by platelets for signal-dependent pre-mRNA splicing and de novo protein synthesis¹⁸⁻²⁸. As a consequence, human platelets retain a group of pre-mRNAs that contain non-coding introns and, in response to activating signals, platelets excise these introns to produce mature messages^{18,20}. These spliced mRNAs are capped and polyadenylated on their 5′- and 3′-ends, respectively, and are therefore translatable^{18,20,25,26}.

To date pre-mRNA splicing events for IL-1β and Tissue Factor (TF) have been characterized using platelets isolated from young, healthy volunteers^{18,20}. In particular, TF pre-mRNA in quiescent platelets is spliced into mature message in a signal-dependent fashion, with TF pre-mRNA splicing and increased TF procoagulant activity being observed within 5 minutes and nearing completion by 1 hour. Pre-mRNA splicing of TF is controlled by cdc2-like kinase 1 (Clk1), an intracellular signaling enzyme that was not known to be present and/or operate in human platelets²⁰. Interruption of Clk1 signaling blocked TF pre-mRNA splicing, protein accumulation and procoagulant activity²⁰. Inhibition of Clk1 activity also prevented platelets from accelerating clot formation in human plasma.

Expression of IL-1β and TF are important in the pathogenesis of coagulation disorders, including sepsis and venous thromboembolism (VTE). Therefore, platelet cells may play a previously unrecognized role in these diseases or disorders.

Sepsis is a common and complex clinical syndrome that results from an injurious constellation of systemic inflammatory host responses to infection^{1,2}. The incidence of sepsis is approximately 3 cases per 1,000 patients per year, translating to an annual burden of approximately 750,000 cases in the U.S.³. The overall mortality associated with sepsis is roughly 30%, rising to 40% in the elderly, and to more than 50% in patients with septic shock³. Sepsis in the United States has an estimated annual healthcare cost of about $17 billion dollars⁶⁵.

Thrombocytopenia is also common in sepsis^{4,5}, occurring in 20-44% of medical and surgical intensive care unit admissions and making sepsis the leading etiology of thrombocytopenia in hospitalized patients⁵⁻⁹. Furthermore, thrombocytopenia and/or a blunted rise in the platelet count are negative prognostic features in patients with sepsis^{5,6,10}.

Disregulated coagulation and the release of cytokines in response to systemic inflammation occurs in sepsis^{4,6} and disseminated intravascular coagulation (DIC), which is common in sepsis, contributes to organ failure and plays a central role in the inflammatory response to severe infection and tissue injury^{4,11}.

Excessive TF production, which is not balanced by TF pathway inhibitor (TFPI), has been associated with a poor prognosis in patients with sepsis²⁹. TF and its effects on the coagulation cascade and subsequent formation and degradation of fibrin clots have been the targets of intense inquiry, and the differential effects of inflammatory cytokines on these variables have been delineated⁴. Activated leukocytes (e.g., monocytes) and endothelial cells modulate coagulant activity and inflammation in patients with sepsis, at least in part, through production of TF and IL-1β^{1,2,6,30-32}.

While platelet adhesion and aggregation have been studied, the observations remain limited and conflicting^{5,4,12-17}. In addition to adhesion and aggregation, stimulus-induced surface translocation and/or release of inflammatory mediators, vascular growth factors and other signaling molecules may be critical in sepsis^{6,15}.

Classification systems such as Acute Physiology Age and Chronic Health Examination (APACHE), and Simplified Acute Physiology Score (SAPS) represent current diagnostics that stratify patients according to physiologic indices. While the diagnostic usefulness of the APACHE II score has been demonstrated, studies have revealed no diagnostic differentiation of sepsis from Systemic Inflammatory Response Syndrome (SIRS) by APACHE II or SAPS II⁶⁵. In addition to physiological systems, biomarkers, such as Tumor Necrosis Factor-α (TNFα), IL-10, IL-1, IL-6, IL-12, IL-18, and CRP have been used or studied as possible diagnostics, however, their utility has fallen short of expectations for various reasons⁶⁵.

Pre-mRNA splicing generated TF also provides a means for disregulation of the coagulation response in the elderly. There are several reports characterizing platelet function in the elderly, and most indicate that platelet reactivity increases with aging.

Enhanced platelet aggregation has been observed in platelet-rich plasma (PRP) in response to adenosine diphosphate (ADP), collagen, and arachidonic acid³³⁻³⁶. Similar aggregation patterns were recorded in whole blood³⁷. Consistent with these responses, increases in thromboxane production have also been found in elderly subjects³⁶. In addition, bleeding times are shortened³⁸ and plasma fibrinogen levels are typically elevated in advanced age³⁷.

Supiano and colleagues³⁹⁻⁴⁴ have also characterized differences in platelet adrenergic responses in the elderly and young. These observations indicate that there is functional uncoupling of the α2-adrenergic receptor-adenylate cyclase complex in elderly humans⁴³, suggesting that regulatory pathways may be altered in an age-dependent fashion. They also demonstrated that platelet alpha-adrenergic responsiveness is enhanced in older hypertensive subjects compared to age-matched normotensive subjects⁴², suggesting that subgroups within the elderly population have variable platelet responses.

Together, these studies indicate that platelet function may differ between the elderly and young, and that there may be differential platelet function between subgroups.

One particular coagulation disorder found in the elderly is venous thromboembolism (VTE), the incidence of which is approximately 10-20 times higher in elderly subjects versus young adults, making advanced age one of the most important risk factors for VTE^{45,46}. The annual incidence of VTE begins rising at age 45 and, is markedly increased after age 65. In addition, survival rates after hospitalization for VTE are lowest in patients older than 70 years^{45,47,48}. Stasis of blood flow, damage to vascular structures, and variations in coagulation responses are underlying factors that increase one's risk for developing VTE³, particularly in the elderly⁴⁶. Malignancy and joint replacement surgeries, which are common in the elderly population, are also risk factors for VTE⁴⁶.

Enhanced diagnostics could foster substantial reductions in sepsis-related mortality, duration of hospitalization and the associated costs. Likewise, prognosis or prediction of VTE could substantially reduce VTE-related mortality, duration of hospitalization and the associated costs. Hence, there is a need in the art for reliable and rapid diagnostic, prognostic and/or predictive methods that identify patients suffering from or likely to develop a coagulation related disease or disorder.

### SUMMARY OF THE INVENTION

The invention relates to diagnostic, prognostic and/or predictive methods comprising measuring pre-mRNA/mature mRNA patterns, Clk1 activation and/or production of functional TF in platelet cells in a subject, and correlating those measurements to a diagnosis, prognosis or prediction of a coagulation disease or disorder. Pre-mRNA/mature mRNA, or RNA splicing, may be measured using TF mRNA and/or IL-1β mRNA.

The invention demonstrates that activation with toxins commonly associated with sepsis, such as MRSA, E. Coli, LPS or alpha-toxin, platelets accelerate TF-dependent coagulation through a process dependent on RNA splicing, which is illustrated by way of TF pre-mRNA splicing. Likewise, Sepsis is also associated with RNA splicing and accelerated TF-dependent pro-coagulant activity. Sepsis patients who spliced TF pre-mRNA were more likely to be severely ill, develop bacteremia, or die before hospital discharge. Thus, the invention provides a method to identify or predict which sepsis patients are at a higher risk of severe sepsis, organ failure, and death.

The invention relates to a diagnostic or prognostic use of pre-mRNA splicing (e.g., TF mRNA and/or IL-1β mRNA), Clk1 activation and/or production of functional TF in platelet cells of a subject, either before surgery, or in response to the physiological stress of surgery, such as an elderly subject undergoing an orthopedic procedure, as an indication of a significantly increased risk (e.g., approximately a 2-fold) for developing VTE (e.g., post-arthroplasty DVT).

The invention also relates to a method of monitoring intensive care patients or other hospital patients that may be predisposed to infections and/or sepsis, where regular blood samples are taken and analyzed for pre-mRNA splicing or TF activation.

The invention also relates to a method for screening a biological sample to detect early stages of infection, SIRS or sepsis comprising the steps of: detecting pre-mRNA splicing by RT-PCR and/or detecting expression of TF on the platelet cell surface by means of flow cytometry and/or monitoring coagulation activity in platelet cells; analyzing the results of the detection; and diagnosing, prognosing and/or predicting a subject outcome based on the pre-mRNA splicing. Preferably, a biological sample is a blood sample. Optionally, measurement of pre-mRNA splicing is subjected to a first analysis that provides a prediction as to the probability of developing sepsis or another coagulation disorder. In one exemplary embodiment, this is expressed as a probability. In an alternative embodiment it is expressed as a binary yes/no result.

The invention also relates to pre-mRNA splicing, Clk1 activation and/or production of functional TF in platelet cells as a biological marker of clinical indices of coagulation and patient outcomes.

In various aspects, the present invention relates to: materials and procedures for identifying or using markers, such as TF pre-mRNA splicing, Clk1 activation or production of functional TF in platelet cells, that are associated with a diagnosis, prognosis, prediction, or treatment of disordered coagulation in a subject; the use of such markers in diagnosing, predicting, prognosing, treating and/or monitoring the course of a treatment regimen in a subject; and using such markers to identify subjects at risk for one or more adverse outcomes related to disordered coagulation.

The invention has particular relevance to conditions characterized by aberrant, unwanted, or otherwise inappropriate blood coagulation, which include, but are not limited to: haemostasis related disorders; hypercoagulate states, including inherited or acquired; thrombosis, including deep vein thrombosis; pulmonary embolism; thromboembolic complications associated with atrial fibrillation; cardiac valve replacement; coronary thrombolysis, for example, during acute myocardial infarction; percutaneous transluminal angioplasty; ischemia-reperfusion injury, post-operative thromboembolism, shock, sepsis, septic shock, toxic shock and systemic inflammatory response syndrome (SIRS).

### BRIEF DESCRIPTION OF THE DRAWINGS

FIGS. 1A, 1B and 1C illustrate TF mRNA expression patterns in platelets isolated from septic patients. TF mRNA species (i.e., spliced or unspliced) were evaluated in freshly-isolated platelets collected from septic patients within the first 24 hours of admission to the ICU (FIG. 1A). In FIG. 1B, the incidence of spliced TF mRNA in patients is subdivided according to APACHE II scores. The asterisk (*) indicates a statistically significant difference (p<0.05) between the incidence of TF pre-mRNA splicing in patients with the lowest versus highest APACHE II scores. FIG. 1C demonstrates that pre-mRNA splicing was more common in elderly patients than patients under the age of 65.

FIG. 2 illustrates that thrombin induces pre-mRNA splicing in the elderly and that Aspirin does not block signal-dependent TF pre-mRNA splicing. Platelets were isolated from an elderly (85 yr) male subject who was taking aspirin (325 mg) daily. The platelets were immediately processed or exposed to thrombin (0.05 U/ml) for 1 hour. TF mRNA expression patterns were subsequently analyzed. As shown in this figure, the TF mRNA species was primarily unspliced in freshly-isolated platelets (baseline) although the mature transcript was visible. In response to thrombin, the majority of pre-mRNA was spliced into mature message.

FIGS. 3A-C demonstrate that platelets activated with fibrinogen and thrombin splice IL-1β pre-mRNA into a mature message and translate the mRNA into protein. FIG. 3A illustrates the IL-1β gene where exon flanking primer sets are color coded to indicate the approximate location of individual PCR reactions that span each intron of the IL-1β gene. FIG. 3B shows the analysis of IL-1β pre-mRNA and mature mRNA in quiescent platelets and in platelets activated by fibrinogen (Fib) for 2 hours in the presence of thrombin (Thr). On the right side, the boxes represent undesignated exons flanking a representative intron to illustrate the patterns of PCR products. FIG. 3C shows immunostaining of actin (green) and IL-1β protein (red) in quiescent platelets and in platelets stimulated with soluble fibrinogen and thrombin for 8 hours. IL-1β protein was detected in platelets that were embedded within fibrin-rich clots, consistent with de novo synthesis of the protein^{18,19}.

FIGS. 4A and 4B illustrate that activated platelets rapidly splice TF pre-mRNA and generate TF-dependent procoagulant activity. FIG. 4A illustrates TF and GAPDH mRNA expression in freshly-isolated platelets (control) and platelets adherent to fibrinogen and co-activated with thrombin (Fib+Thr). pHTF=pre-mRNA for human tissue factor; mHTF=mRNA for human tissue factor. FIG. 4B shows a time course (0-60 min) of TF-dependent procoagulant activity in platelets that have adhered to fibrinogen in the presence of thrombin. The lines represent the mean±SEM of three independent experiments and the asterisk (*) indicates a statistically significant difference (p<0.05) between freshly-isolated and activated platelets²⁰.

FIG. 5 shows that inhibition of Clk1 activity in activated platelets reduces clot formation. Platelets were left quiescent or activated with thrombin for 2 hours in the presence or absence of the Clk inhibitor (Clk Inh), which blocks pre-mRNA splicing, and plasma clot formation was measured as described11. The bars represent the mean±SEM of 5 independent experiments and the asterisk (*) indicates a statistically significant difference (p<0.05) in the rate of clot formation in plasma samples exposed to activated platelets compared with quiescent or treated platelets. The anti-TF bar represents activated platelets treated with a neutralizing antibody directed against TF. Similar results were observed in platelets that were activated for 5 minutes²⁰.

FIG. 6 shows TF mRNA expression in platelets isolated from elderly and young subjects. TF mRNA expression patterns were evaluated in freshly-isolated platelets from two young (<40 yrs) and two elderly (65, 89 yrs) subjects. The two young subjects were not medicated. Both the 65-year old (lane 3) and the 89-year old (lane 4) subjects were taking aspirin but no other prescribed medications. The elderly subjects were both males.

FIG. 7 illustrates the TF-dependent procoagulant activity in freshly-isolated platelets from elderly and young subjects. TF-dependent procoagulant activity was evaluated in freshly-isolated platelets from eight young (<40 yrs) and five elderly (65-79 yrs) donors. The eight young subjects were not medicated. One of the high-responding elderly subjects was on aspirin alone while the other was not medicated. Of the three remaining elderly subjects, one was on aspirin alone, one was treated with coumadin and an anti-hypertensive, and one was not medicated. The elderly and young subjects consisted of male and female donors.

FIG. 8 shows TF mRNA expression patterns in platelets isolated from septic patients. TF mRNA species were evaluated in freshly-isolated platelets collected from septic patients or healthy volunteers. For the septic patients, the platelets were isolated within 24 hours of admission to the ICU. The left panel shows a septic patient whose platelets express unspliced, pre-mRNA for TF. The middle panel shows a septic patient whose platelets express unspliced and spliced TF mRNA species (i.e., partially spliced). The right panel shows a septic patient whose platelets express TF transcripts that are completely spliced. Platelets from the healthy volunteers expressed unspliced, TF pre-mRNA.

FIG. 9 shows that TF-dependent procoagulant activity is increased in freshly-isolated platelets obtained from septic patients compared to healthy volunteers. TF-dependent procoagulant activity associated with platelets from each septic subject was higher than activity associated with platelets from the healthy volunteer that was assayed in parallel.

FIG. 10 shows TF mRNA expression patterns in platelets isolated serially from septic patients. FIG. 10A shows TF mRNA species that were evaluated in a septic patient during hospitalization. FIG. 10B illustrates the number of patients whose platelets expressed spliced TF mRNA at some point during hospitalization.

FIGS. 11A and 11B demonstrate that LPS induces TF pre-mRNA splicing in platelets. FIG. 11A shows TF mRNA expression patterns in platelets that were left quiescent or activated with LPS (10 ng/ml) for 2 hours. In data not shown, LPS induces pre-mRNA splicing in platelets within 5 minutes. In FIG. 11B platelets were stimulated with LPS for 2 hours in the presence or absence of the Clk1 inhibitor (Clk Inh) The bars represent the mean±SEM of 4 independent experiments and the asterisk (*) indicates a statistically significant difference (p<0.05) in the rate of clot formation in plasma samples exposed to activated platelets compared with quiescent or treated platelets. The anti-TF bar represents activated platelets treated with a neutralizing antibody directed against TF.

FIGS. 12A and 12B demonstrate that α-toxin induces TF pre-mRNA splicing in platelets. FIG. 12A shows the TF mRNA expression patterns in platelets that were left quiescent or activated with α-toxin (10 ng/ml) for 2 hours. In FIG. 12B platelets were stimulated with α-toxin for 2 hours in the presence or absence of the Clk1 inhibitor (Clk Inh) The bars represent the mean±SEM of 4 independent experiments and the asterisk (*) indicates a statistically significant difference (p<0.05) in the rate of clot formation in plasma samples exposed to activated platelets compared with quiescent or treated platelets. The anti-TF bar represents activated platelets treated with a neutralizing antibody directed against TF.

FIG. 13 demonstrates that S. aureus incubated with whole blood induces TF pre-mRNA splicing in platelets. Methicillin Sensitive S. aureus (MSSA) was cultured from the bloodstream of a septic patient and incubated with whole blood. After 240 minutes, the platelets were isolated from the whole blood and TF pre-mRNA splicing was assessed. Control identifies whole blood that was left untreated. pHTF=pre-mRNA for human tissue factor; mHTF=spliced mRNA for human tissue factor. Similar results were observed with E. coli (data not shown).

FIG. 14 demonstrates that platelets generate TF-dependent procoagulant activity in response to thrombin. Platelets and monocytes were isolated from the same donor and incubated with thrombin (0.05 U/ml) for the designated times. The average number of platelets (1.1±0.6×10⁹) and monocytes (1.8±0.2×10⁶) used for these studies were based on the number of cells present in 5 ml of whole blood as measured by a National Reference Laboratory (ARUP). For these studies, the average circulating cell counts per μl of whole blood were 211,667±23,412/μl and 367±33/μl for platelets and monocytes, respectively; these values fall within the normal range for each cell. The data are graphed as fold increases in TF-dependent procoagulant activity over baseline and the bars represent the mean±SEM for 3 independent experiments.

FIG. 15 demonstrates that platelets and monocytes generate TF-dependent procoagulant activity in response to LPS. For each experiment, platelets and monocytes were isolated from the same donor and incubated with LPS (10 ng/ml) for the designated times. Cells used for this analysis were from the same subjects who were studied in FIG. 6. The data are graphed as fold increases in TF-dependent procoagulant activity over baseline and the bars represent the mean±SEM for 3 independent experiments.

FIG. 16 illustrates blood draw time points for an exemplary embodiment of the invention.

FIG. 17 illustrates blood draw time points for an exemplary embodiment of the invention.

### BEST MODE(S) FOR CARRYING OUT THE INVENTION

As used herein, “blood” means whole blood or any fraction thereof, for example plasma, platelets, and a concentrated suspension of cells.

As used herein, “detectable moiety” or a “label” refers to a compound or composition detectable by spectroscopic, photochemical, biochemical, immunochemical, or chemical means. For example, useful labels include, but are not limited to, ³²P, ³⁵S, fluorescent dyes, electron-dense reagents, enzymes, biotin-streptavadin, dioxigenin, haptens and proteins.

As used herein, “disease prediction,” “prediction” or similar terms, means to predict the occurrence of disease before it occurs.

As used herein, “diagnosis” or “diagnostic” means a prediction of the type of disease or condition from a set of marker values and/or patient symptoms.

As used herein, “disordered coagulation” includes, but is not limited to, thromboembolic disease, intravascular thrombosis, microvascular platelet thrombosis, venous thromboembolism, deep vein thrombosis, disseminated intravascular coagulation (DIC), coronary artery disease, fibrinolysis, and/or sepsis.

As used herein, “prognosis” or “prognostic”, means to predict disease progression at a future point in time from one or more indicator values.

As used herein, “sample” means any sample of biological material derived from a subject, such as, but not limited to, blood, plasma, mucus, biopsy specimens and fluid, which has been removed from the body of the subject. The sample which is tested according to the method of the present invention may be tested directly or indirectly and may require some form of treatment prior to testing. For example, a blood sample may require one or more separation steps prior to testing. Further, to the extent that the biological sample is not in liquid form, (for example it may be a solid, semi-solid or a dehydrated liquid sample) it may require the addition of a reagent, such as a buffer, to mobilize the sample.

As used herein, “subject” means a mammal, including, but not limited to, a human, horse, bovine, dog, or cat.

As used herein, “platelets” or “platelet cells” means a preparation enriched for platelet cells, microparticles, or a combination thereof.

As used herein, “TF pre-mRNA splicing” means signal dependent removal of at least one intronic sequence from a pre-existing RNA within a platelet cell, and preferably removal of all intronic sequences so as to produce a mature mRNA capable of being translated into TF protein. TF pre-mRNA splicing may be measured directly, for example, by PCR, or indirectly, for example, by measuring TF-dependent coagulation activity, TF protein production or Clk1 activation.

As used herein, “venous thromboembolic (VTE)” is used to describe a blood clot (thrombus) in a major vein and includes microvascular platelet thrombosis, pulmonary embolism (PE) or pulmonary thromboembolism (PTE) and Deep vein thrombosis (DVT).

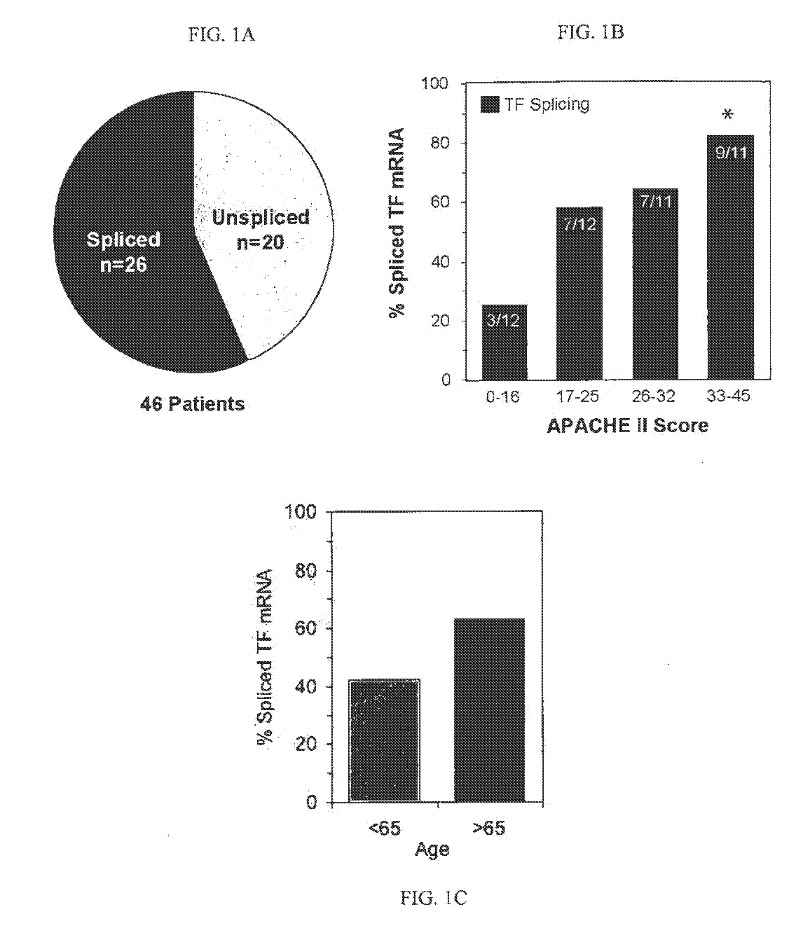

The invention relates to the finding that platelets from healthy human subjects contain TF pre-mRNA and process it to the mature transcript in response to cellular activation. As a result, activated platelets produce TF protein, have procoagulant activity, and accelerate plasma clot formation. The intracellular signaling pathway that controls TF pre-mRNA splicing has been found to involve a Cdc2-like kinase, Clk1, an enzyme present in platelets Inhibition of Clk1 signaling in activated platelets blocks splicing factor 2 (SF2)/alternative splicing factor (ASF) phosphorylation, TF pre-mRNA splicing, and de novo accumulation of bioactive TF protein. Hence, Clk1-dependent splicing of TF pre-mRNA in platelets leads to fibrin formation and stabilization of a platelet thrombus. Furthermore, TF pre-mRNA splicing in septic and elderly subjects demonstrate that some of these activities change in a pro-thrombotic disease state³² and the elderly (see, FIG. 1).

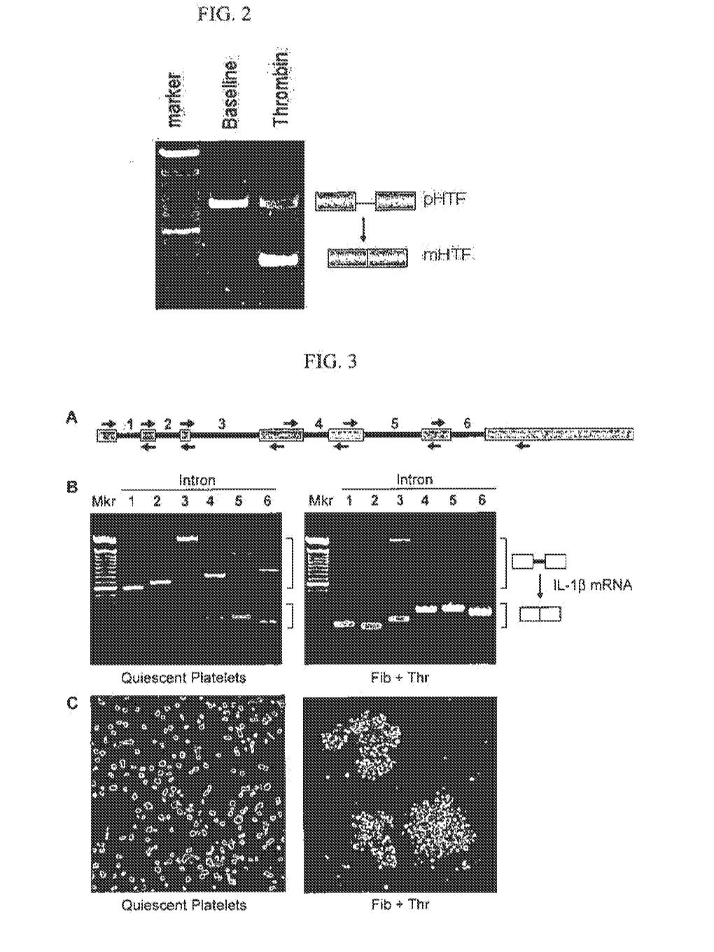

Platelets from elderly patients respond to thrombin by splicing IL-1β and TF pre-mRNA into mature, translatable mRNA (see, FIG. 2). Therefore, other agonists, such as platelet-activating factor (PAF), ADP and collagen are also expected to induce pre-mRNA splicing and associated protein synthesis in the elderly²⁰. Therefore, thrombin and/or fibrinogen are used herein merely as an example of a platelet-activating factor.

The present invention demonstrates that pre-mRNA splicing is markedly increased in patients with sepsis, a clinical condition in which disordered coagulation is a central feature³². Within the first 24 hours of admission into the intensive care unit (ICU), it was found that platelets from over half of the patients expressed spliced TF mRNA and that the incidence of splicing was increased in patients with high APACHE II scores, an index of the severity of critical illness (FIG. 1). While the APACHE II scale is most accurate during the seven days immediately prior to the death of a patient, the present invention may provide diagnostic, prognostic or predictive indices over a substantially longer time course or may provide an earlier diagnostic, prognostic or predictive index than current tests, such as APACHE II.

In addition, the present invention demonstrates that pre-mRNA splicing was more common in elderly patients than patients under the age of 65 (FIG. 1C). Because some of these patients received heparin to prevent VTE, it also appears that heparin does not block TF pre-mRNA splicing. This indicates that platelets from elderly subjects may have an enhanced predisposition for activation of the TF pre-mRNA splicing pathway compared to platelets from young donors.

In an exemplary embodiment, the invention relates to the use of TF pre-mRNA splicing in a platelet cell as an indicator, prognostication or diagnostic for disordered coagulation, such as sepsis, VTE and/or DVT. In another exemplary embodiment, the invention relates to predicting an increased probability of VTE in elderly patients having an elevated level of TF pre-mRNA splicing prior to undergoing a surgery, such as an orthopedic surgery.

A standard method for characterizing diagnostic utility is the ROC curve, which plots the sensitivity (true-positive diagnoses) of a diagnostic marker at a specified value against the specificity (false-positive diagnoses). APACHE (Acute Physiology and Chronic Health Evaluation) II, SOFA (Sepsis-related Organ Failure Assessment), and SAPS (Simplified Acute Physiology Score) II scores are measurements of illness severity calculated from clinical and laboratory parameters, which correlate well with mortality in septic patients⁴⁹⁻⁵¹.

The presence of mature, spliced TF mRNA may provide clinically-relevant information, particularly for sepsis and in the elderly, that has disease predictive, diagnostic, or prognostic value, similar to a recent observation of predictive information based on the presence of myeloid-related protein-14 (MRP-14) transcripts in platelets from subjects with coronary artery disease and ST-segment elevation myocardial infarction (STEMI)⁵¹. Thus, the invention provides a biological marker useful as a diagnostic, prognostic or for disease prediction of patient health, e.g., increased risk of mortality in sepsis or increased risk of a coagulation disorder in the elderly.

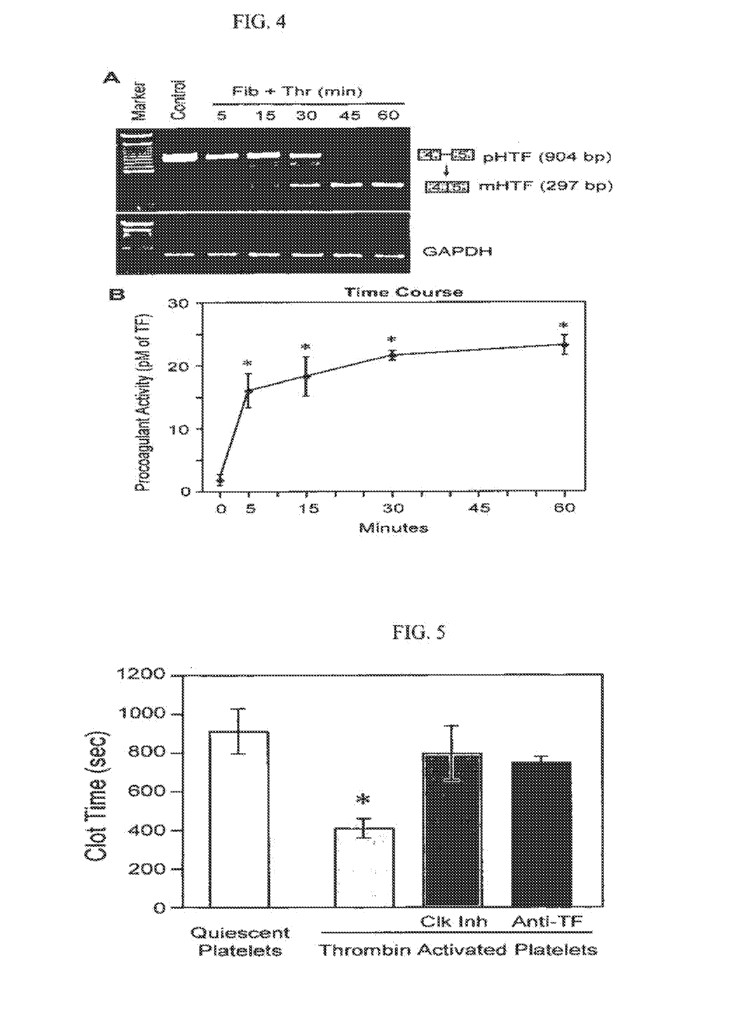

Expression of unspliced, TF pre-mRNA may also be used to predict improved survival (e.g., in sepsis) because newly released platelets from the bone marrow are no longer being activated by inflammatory agonists present in the circulation. Similar to TF pre-mRNA splicing, IL-1β pre-mRNA splicing is associated with increased expression of intracellular IL-1β protein in platelets (see, FIG. 3). Thus, the invention also relates to the use of IL-1β pre-mRNA splicing as a diagnostic, prognostic, or for disease prediction of a coagulation disorder or disease. Hence, while the invention is described in terms of TF pre-mRNA, it is to be understood that the invention also relates to IL-1β.

According to the invention, at least one substance can be used for detecting the expression and/or function of Clk1, TF protein, TF-dependent coagulation activity, mature TF mRNA and/or TF pre-mRNA in or associated with platelet cells. This also makes it possible to provide a diagnosis, prognosis, or to predict diseases which are connected with a disturbed activity of TF. For example, an antibody which is directed against Clk1 and/or TF may be employed in a detection method, such as ELISA (enzyme-linked-immuno sorbent assay), which is known to the skilled person. Other substances that may be used for the diagnostic detection are oligonucleotides, which are suitable, for example, using the polymerase chain reaction (PCR), for detection of mature TF mRNA and/or TF pre-mRNA, either with or without amplification of the RNA or cDNA to be analyzed. Yet other substances that may be used for the diagnostic detection are polypeptides, including antibodies, which are suitable for detection of CLk1 activity or activation, or production of TF protein (e.g., by ELISA or Western Blot). Alternatively, TF-dependent coagulation activity may be measured in a sample obtained from an appropriate subject.

The invention also relates to a diagnostic kit. This kit comprises at least one substance which is suitable for detecting the expression and/or function of Clk1, TF protein, TF-dependent coagulation activity, mature TF mRNA and/or TF pre-mRNA in platelet cells, for the purpose of diagnosing, prognosing, or predicting diseases which are connected to a disordered coagulation. The diagnostic kit according to the invention comprising a substance for detecting the expression and/or function of Clk1, TF protein, TF-dependent coagulation activity, mature TF mRNA and/or TF pre-mRNA in platelet cells, additional assay components (e.g., reagents), labels, and/or instructions.

In an exemplary embodiment, the invention provides a method or kit for detecting the activation of TF in platelet cells, for example, by measuring TF RNA splicing, either directly or indirectly, for example, by measuring TF-dependent coagulation activity or Clk1 activity, wherein a biological sample, such as a blood sample, is withdrawn from a subject, platelet cells and/or microparticles (a purified cell preparation) are purified from the biological sample, TF splicing is measured in the purified cell preparation, and the degree of TF splicing is correlated with a diagnosis/prognosis/prediction for a coagulation related disease, such as sepsis, VTE, or DVT.

In an exemplary embodiment, the splicing incidence for septic patients may be approximately 50% (see, FIG. 1A) in the first 24 hours after admission to the ICU, the splicing incidence for hospitalized non-septic patients may be approximately 25%, based on the fact that heart failure is associated with platelet abnormalities and increased risk for venous thromboembolism^{52,53}, and the splicing incidence may be approximately 0% for healthy volunteers, based on findings in 54 normal subjects whose platelets exclusively expressed unspliced, TF pre-mRNA.

In another exemplary embodiment, it is anticipated that approximately one third of patients undergoing joint replacement surgery will have spliced pre-mRNA in the pre- or post-operative period while approximately two-thirds will not. Those expressing spliced TF mRNA may benefit from coagulation therapy even in the absence of other disease indications. In another exemplary embodiment, subjects expressing spliced pre-mRNA prior to surgery will have an elevated risk of developing VTE and may benefit from coagulation therapy prior to and/or following surgery, with or without a diagnosis of VTE.

The invention demonstrates that TF pre-mRNA splicing and associated protein responses are increased in freshly-isolated platelets from a subset of elderly subjects, compared to young volunteers. The increased pre-mRNA splicing in freshly-isolated platelets from elderly subjects are assayed to confirm the correlation with increased baseline Clk1 activity. Thus, pre-mRNA splicing provides a previously-unrecognized marker of platelet-mediated procoagulant and inflammatory activity in the elderly. In an exemplary embodiment, a robust correlation between clinical coagulation indices (e.g., D-dimers, PTT, PT, etc.) and the expression of spliced TF mRNA in freshly-isolated platelets from the elderly provides a valuable diagnostic or prognostic indicator, for example, to identify elderly subjects who are at increased risk for VTE and other coagulation disorders.

TF-dependent coagulation activity may be measured by any method known in the art, including, but not limited to the Actichrome TF assay (available from America Diagnostica, Inc.). This assay measures the peptidyl activity of human tissue factor in cell lysates and human plasma. Samples are mixed with human factor VIIa and human factor X. The reagents are incubated at 37° C., allowing for the formation of the tissue factor/factor VIIa complex (TF/FVIIa) complex and conversion of human factor X to Factor Xa by the complex. Factor Xa is measured by its ability to cleave Spectrozyme® Xa, a chromogenic substrate. Absorbance is read at 405 nm and compared to values obtained from a standard curve of known amounts of active human tissue factor.

TF pre-mRNA splicing may be measured by any method known in the art, including, but not limited to, PCR using primers that target sequences in exon four (5′-CTCGGACAGCCAACAATTCAG-3′; SEQ ID NO: 1) and five (5′-CGGGCTGTCTGTACTCTTCC-3′; SEQ ID NO: 2), and thus span intron four³. Likewise, detection of full-length mature mRNA for human TF (mHTF) in platelets may be measured using any method known in the art, including, but not limited to, using primers targeting sequences in exon one (5′-CCAACTGGTAGACATGGAGAC-3′; SEQ ID NO: 3) and exon six (5′-CAGTAGCTCCAACAGTGCTTCC-3′; SEQ ID NO: 4). In light of the disclosure herein, a person of ordinary skill in the art may select and use alternative primers based on the sequence of the gene/RNA⁵⁴. Primer design may be aided by the use of available computer programs, such as OLIGO™ (available from Hitachi Software), Primer3 (available online from the University of Massachusetts Medical School), GeneFisher (available online from the Universitat Bielefeld, Germany), or OligoAnalyzer (available from Integrated DNA Technologies, Inc.).

Indirect in situ hybridization or direct in situ PCR may be used to detect TF pre-mRNA in megakaryocytes and platelets¹⁸. Primers specific for intron four (5′-ACCCATTTCTTCCCCAATTC-3′ (SEQ ID NO: 5) and 5′-GTGCCTGGGATCCTCAATAG-3′ (SEQ ID NO: 6) were used to generate DIG-labeled intronic probes for the indirect in situ PCR and direct in situ PCR experiments. For platelets that were adherent to fibrinogen in the presence of thrombin, the generated cDNA was amplified in the presence of DIG-labeled dNTP using primers that targeted exons three (5′-CTCCCCAGAGTTCACACCTTAC-3′; SEQ ID NO: 7) and five (5′-CGGGCTGTCTGTACTCTTCC-3′; SEQ ID NO: 8), respectively. These exonic primers allowed detection of the spliced product (331 bp), but not the unspliced product (3,635 bp), by normal PCR methods.

TF-dependent procoagulant activity may be measured using any method known in the art, including, but not limited to, an Actichrome TF assay (American Diagnostica)⁵⁵. For the experiments described herein a total of 2×10⁹ freshly isolated CD45-depleted platelets, a value that approximates the number of platelets found in 10 ml of whole blood obtained from healthy subjects, were resuspended in M199 media. Platelets were left quiescent or activated in the presence or absence of Tg003. At the end of each experimental point, the platelets were immediately centrifuged at 15,500 g for 4 min at 4° C. The supernatants were collected and recentrifuged at 100,000 g for 90 min at 4° C. to pellet microparticles. In parallel, the platelet pellets were resuspended in ice-cold 250 mM sucrose that was suspended in 10 mM of PBS that contained a broad band protease inhibitor cocktail. After a brief sonication to disrupt the cells, the platelets were centrifuged for 15 min at 420 g (4° C.) to separate the sedimented cellular components from the supernatant-rich membranes. The supernatants were recentrifuged at 20,800 g for 30 min (4° C.) to pellet the membrane proteins. Intact cellular membranes and microparticles were immediately placed in 25 μl of kit assay buffer and TF-dependent procoagulant activity was calculated. In separate studies, disruption of platelet membranes or microparticles by standard detergent lysis was found to markedly reduce activity. To demonstrate the specificity of the assay for TF procoagulant activity, some samples were preincubated with a neutralizing TF antibody (pAb 4502; American Diagnostica). Factor VIIa was also eliminated from the reaction. The data shown herein are generally displayed as pM of TF per 2×10⁹ platelets.

Clk1 activity in platelets may be measured by any method know in the art. In an exemplary embodiment, Clk1 activity may be determined using an immune complex kinase assay. An antibody against Clk1 is used for immunoprecipitation of the protein. Nonimmune rabbit IgG is used as a control, and in select experiments recombinant SF2/ASF is removed from the assays to screen for nonspecific incorporation of radiolabeled phosphate. Kinase assays are performed by addition of recombinant SF2/ASF (Protein One) in the presence of γ-[³²P]ATP (MP Biomedicals). At the end of this incubation period, the agarose beads and immune complexes are removed by centrifugation, and the unbound sample, which contained SF2/ASF, is resolved by SDS-PAGE.

For additional methodologies, see U.S. Pat. No. 7,045,289, and U.S. Patent Publications 20040197845, and 20060078559.

A sample may be measured for TF pre-mRNA splicing, either directly or indirectly, and the results compared to a standard sample. A difference of about 10% or more, about 15% or more, about 20% or more, about 25% or more, about 30% or more, about 35% or more, about 40% or more, about 45% or more, about 50% or more, about 55% or more, about 60% or more, about 65% or more, or about 70% or more, in the activity measured in any of these assays, relative to the activity of the standard, is diagnostic for a disease or disorder characterized by disordered coagulation.

Tg003 ((Z)-1-(3-Ethyl-5-methoxy-2,3-dihydrobenzothiazol-2-ylidene)propan-2-one) is an example of a Clk1 inhibitor that is commercially available. It is a cell-permeable dihydrobenzothiazolo compound reported to be a potent, specific, reversible, and ATP-competitive inhibitor of Clk-family of kinases (Ki=10 nM for mClk1/Sty; IC50=15 nM, 20 nM, 200 nM, and >10 μM for mClk4, mClk1, mClk2, and mClk3, respectively). It does not affect the activities of SRPK1, SRPK2, PKA, or PKC at concentrations up to 1 μM. See U.S. Patent Pub. 20050171026, which is incorporated by reference.

Purified platelets and possibly monocytes may be centrifuged to obtain cellular pellets and supernatants. The supernatants may be re-centrifuged to obtain microparticles and microparticle-free medium. Pro-IL-1β, mature IL-1β, and TF protein may be measured in the cell supernatant. RANTES accumulation in the platelet supernatants may also be measured as an index of platelet activation^{23,56}. Pro-IL-1β, mature IL-1β, and TF-dependent procoagulant activity may also be measured in the platelets, monocytes, and/or microparticles. The cell pellets may be used to assess TF mRNA expression patterns and intracellular protein accumulation in the purified platelets and monocytes. In addition, cell-associated pro- and mature IL-1β protein and TF-dependent procoagulant activity are measured¹⁸⁻²⁰.

### Example I

To determine if differences exist in the response of platelets from the elderly and young, platelet-derived TF activity was measured in elderly subjects. Elderly subjects (65 or older) consisted of non-medicated volunteers, who were otherwise healthy, as well as subjects who were medicated.

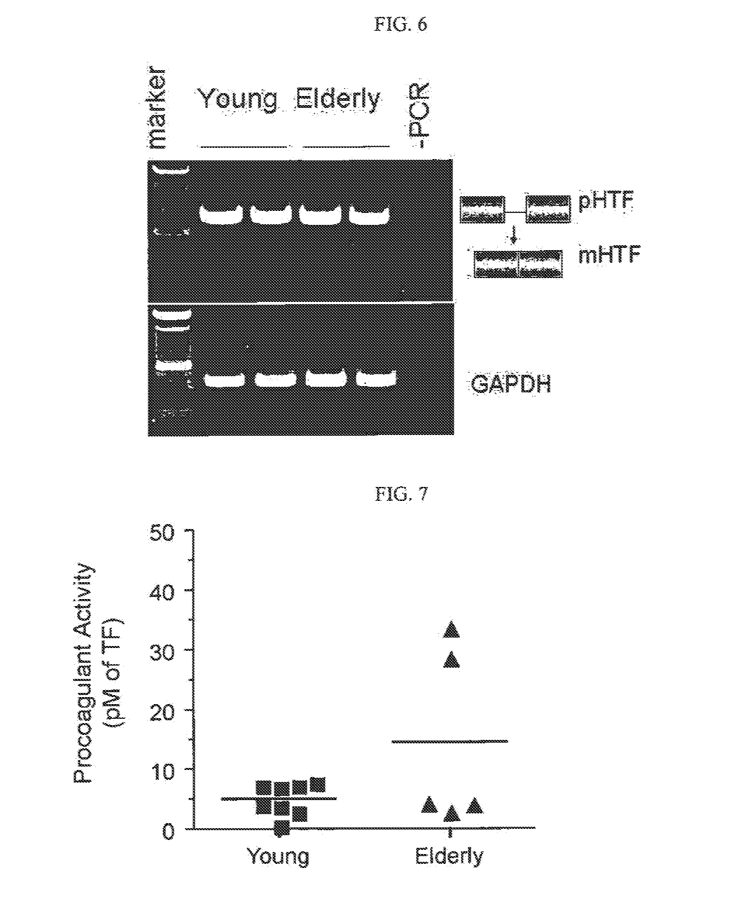

A consistent observation in young healthy donors (n=54) is that the TF mRNA species in freshly-isolated platelets is an unspliced, pre-mRNA (see, FIGS. 4A, 6, and 8). To assess the question of whether or not similar patterns exist in platelets isolated from elderly subjects, the TF mRNA species in freshly-isolated platelets from two elderly subjects were compared to patterns in young subjects. Platelets from all of the subjects expressed TF pre-mRNA and GAPDH (FIG. 6). However, one of the two elderly patients also expressed spliced TF mRNA at baseline without evidence of any acute illness (FIG. 6).

Increased detection of spliced TF mRNA in freshly-isolated platelets from elderly subjects suggests that TF-dependent procoagulant activity may be increased in the elderly. Therefore, additional elderly and young subjects were recruited and TF-dependent procoagulant activity was measured in freshly-isolated platelets from these subjects. Consistent with previous observations, TF-dependent procoagulant activity was very low in resting platelets that were freshly-isolated from young donors (FIG. 7). In contrast, TF-dependent procoagulant activity was markedly elevated in platelets isolated from two of the five elderly donors (FIG. 7). These results indicate that circulating platelets from some elderly subjects have increased TF pre-mRNA splicing and high TF activity and that identification of such individuals may be important in assessing their risk for developing VTE and other coagulation related diseases or disorders.

### Example II

Without exception (n=54), the TF mRNA species in freshly-isolated platelets from young healthy volunteers (<40 yr) is an unspliced, pre-mRNA (FIGS. 5, 7, 10). Patients with sepsis were identified in the University of Utah Intensive Care Unit (ICU) using consensus criteria^{3,57}. Table 1 summarizes the demographic data for this study.

TABLE 1

| Demographic | |
|---|---|
| Number of Patients | 46 |
| Mean Age | 57 ± 17 years |
| Male | 54% |
| Mean APACHE II Score | 25.6 ± 9.2 |
| Mean SAPS Score | 14.7 ± 4.9 |
| Means SOFA Score | 8.5 ± 4 |
| Bacteremia | 52% |
| Documented Bacteremia | 38.9% |
| Platelet count (K/μl) | 232 ± 136 |
| WBC (K/μl) | 17.8 ± 10.3 |
| ICU LOS | 6.7 ± 9.7 days |
| Mortality | 34.5% |
| In-Hospital Mortality | 13% |

Platelets were isolated from each patient within 48 hours of admission to the ICU and TF mRNA expression patterns were characterized in each patient. In addition, clinical data for each patient including age and gender, admission diagnoses, APACHE II score, laboratory and microbiology results, and mortality were also collected. When feasible, platelets were also isolated from blood that was obtained on days three, five, and ten. The blood samples were processed immediately. Pre-mRNA splicing was also evaluated in platelets isolated from healthy volunteers (age 18-50) that were not taking medications. Platelets isolated from patients with sepsis and routine healthy control donors were processed in parallel.

Summary data and patient demographics are expressed as mean±SD. Continuous variables were assessed for normality and if distribution was normal, parametric t-tests were used. If distributions were not normal, Wilcoxon Rank Sum tests were used. Categorical variables were compared using the Fisher's Exact test. Logistic regression analyses were used to determine odds ratios (OR).

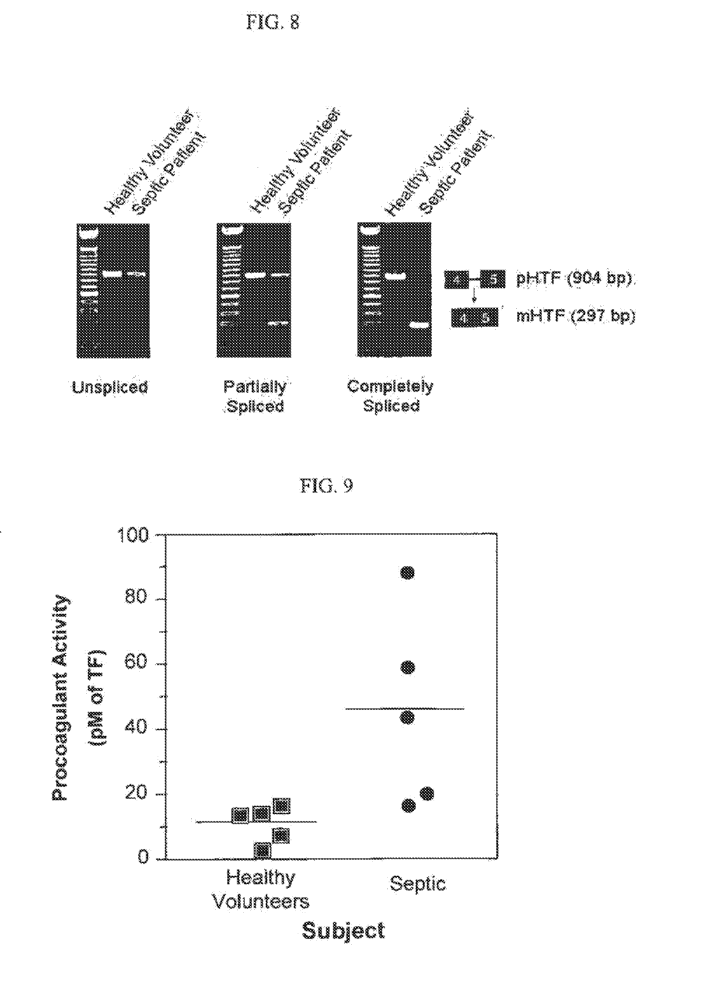

Three patterns of TF mRNA expression were identified (FIG. 8) in septic patients: 1) mRNA that was unspliced (pre-mRNA only); 2) mRNA that was partially spliced (both pre-mRNA and spliced mRNA species are present); and 3) mRNA that was completely spliced (mature mRNA only).

To simplify the mRNA expression analysis, the partially spliced and completely spliced mRNA patterns were grouped together to delineate the number of septic patients whose platelets expressed a processed TF mRNA species. Platelets from over half of the septic patients expressed spliced TF mRNA within 48 hours of admission (FIG. 1A), and the incidence of splicing was increased in patients with higher APACHE II scores (FIG. 1B), a commonly used index of critical illness^{3,57}. In addition, the incidence of mortality more than doubled (2.6 fold) in patients whose platelets expressed spliced TF mRNA compared to those who did not.

This data suggested that platelets isolated from the systemic circulation of septic patients may have increased TF-dependent procoagulant activity compared to platelets isolated from healthy volunteers. Therefore, five additional patients and healthy volunteers were recruited and the TF-dependent procoagulant activity in their platelets measured using an Actichrome® TF assay. Platelets from the septic patients were isolated within 24 hours of admission to the ICU. TF-dependent procoagulant activity was increased in platelets isolated from septic patients compared to healthy donors (FIG. 9). Because all of the platelets in the samples from these septic patients were used for the functional assays, TF pre-mRNA splicing was not experimentally confirmed. In these studies, TF activity was increased in platelet membranes isolated from patients with sepsis compared to healthy controls. The mean pM of TF for control donors was 11.12±6.25 and for patients with sepsis was 45.89±29.6 (p=0.03).

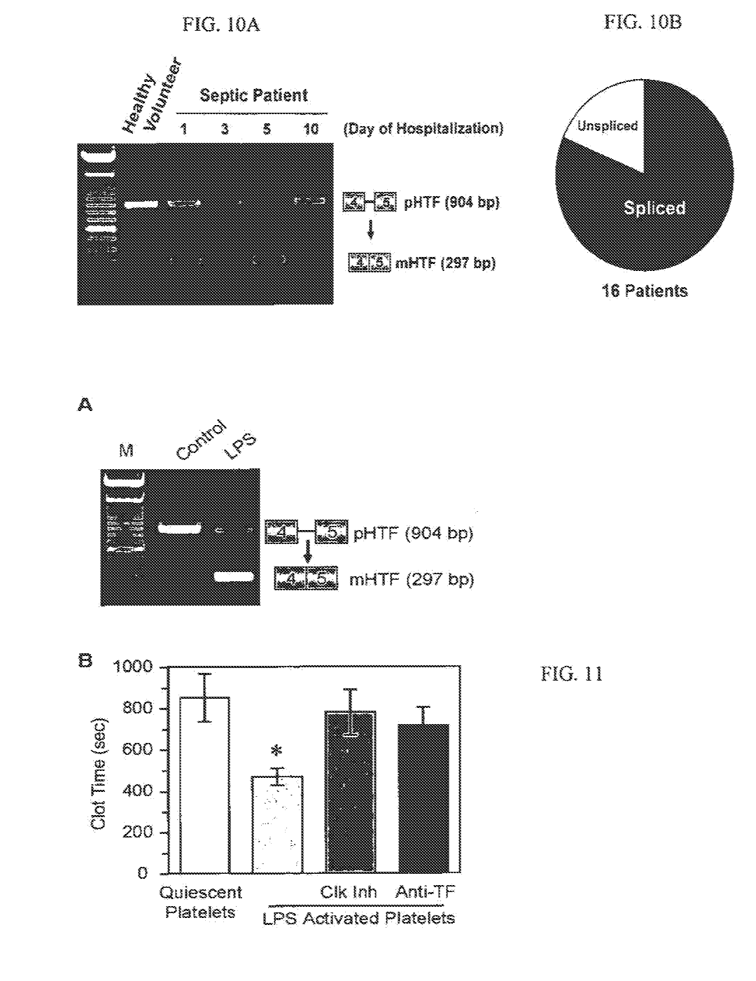

Further, TF pre-mRNA splicing patterns were measured in serial blood samples collected from 16 patients during their stays in the ICU. FIG. 10A shows an example of pre-mRNA splicing patterns in one patient. The pie chart in FIG. 10B represents the number of patients whose platelets expressed spliced TF mRNA at any time during their stay in the ICU. The data indicate that spliced TF mRNA is present in platelets from most patients with sepsis at some point during their illness, consistent with an enhanced procoagulant phenotype (FIG. 9), and that new platelets containing unspliced pre-mRNA enter the circulation at later stages in patients that survive. Based on these results, it is anticipated that early detection of spliced TF mRNA indicates an approximately 2× increase in the risk of mortality for that subject.

Overall, 52% of the 46 patients with sepsis expressed mature, spliced TF mRNA (Table 2). In comparison, platelets isolated from over 50 control donors expressed unspliced TF pre-mRNA. In a subset of 15 sepsis patients who had serial blood samples drawn, 80% expressed mature spliced TF mRNA on at least one day during the first five days of their MICU course. A representative example of serial blood draws from a patient with sepsis over a ten day period is shown in FIG. 10A. The pattern shows progression from partially spliced TF mRNA to completely spliced mRNA on day 5. Isolated platelets from the same patient on day 10 demonstrate only the unspliced TF pre-mRNA form. In this patient, expression of TF pre-mRNA on day 10 paralleled resolution from their sepsis.

Sepsis patients who were ≧65 years, had an APACHE II score upon MICU admission >20, or with bacteremia were more likely to express mature, platelet-derived TF mRNA. In addition, patients with sepsis who expressed mature platelet-derived TF mRNA were more likely to die prior to hospital discharge. These differences persisted after adjusting for age.

Since patients with sepsis are often most severely ill during the initial course of their illness, a subgroup analysis was performed on blood samples drawn within the first 72 hours of enrollment into the study. Within this subgroup, 49% (n=22/45, one patient's sample was not analyzable) of isolated platelet samples from patients with sepsis demonstrated TF pre-mRNA splicing. The odds of expressing spliced TF mRNA in platelets isolated from these sepsis patients increased with age ≧65 (OR 3.76, 95% CI 0.81, 20.90) and APACHE II score >20 (OR 4.21, 95% CI 0.85, 28.68).

TABLE 2
Comparison of Patients with and without Tissue Factor Splicing Phenotype
from Platelets Isolated in Sepsis Patients

| | | | | | | | Odds Ratio | |
|---|---|---|---|---|---|---|---|---|
| | | | | | | | (95% CI) | |
| | | | | | Odds Ratio | | Adjusted for | |
| | +TF splicing | | −TF splicing | | (95% CI) | | Age | |
| Number of | 25 | | 21 | | — | | — | |
| patients | | | | | | | | |
| Age ≧65 | 10 | (40%) | 4 | (19%) | 2.77 | (0.63, 14.74) | — | |
| Female^{†} | 12 | (48%) | 9 | (43%) | 1.23 | (0.33, 4.64) | 1.40 | (0.37, 5.66) |
| APACHE II > 20* | 21 | (84%) | 11 | (52%) | 4.60 | (1.03, 25.02) | 4.06 | (0.87, 22.69) |
| Bacteremia^{β} | 15 | (63%) | 8 | (37%) | 2.45 | (0.63, 10.11) | 2.45 | (0.63, 10.11) |
| D-dimer >8.2 | 6/19 | (32%) | 3/10 | (30%) | 1.34 | (0.65, 2.91) | 1.32 | (0.64, 2.83) |
| [units]^{‡} | | | | | | | | |
| MICU LOS | 8.3 | | 4.8 | | 1.05 | (0.97, 1.17) | 1.06 | (0.98, 1.18) |
| (days)* | | | | | | | | |
| Survival to | 20 | (80%) | 20 | (95%) | 0.21 | (0.004, 2.08) | 0.23 | (0.004, 2.40) |
| Hospital | | | | | | | | |
| Discharge | | | | | | | | |

*mean (95% CI);
^{†}number (%);
^{‡}D-dimer levels were only available for 29 of patients upon MICU admission and study enrollment;
^{β}Culture data on 2 patients was unavailable.
Abbreviations:
APACHE II: Acute Physiology and Chronic Health Evaluation II;
WBC: White Blood Cells;
MICU: Medical Intensive Care Unit;
LOS: Length of Stay

### Example III

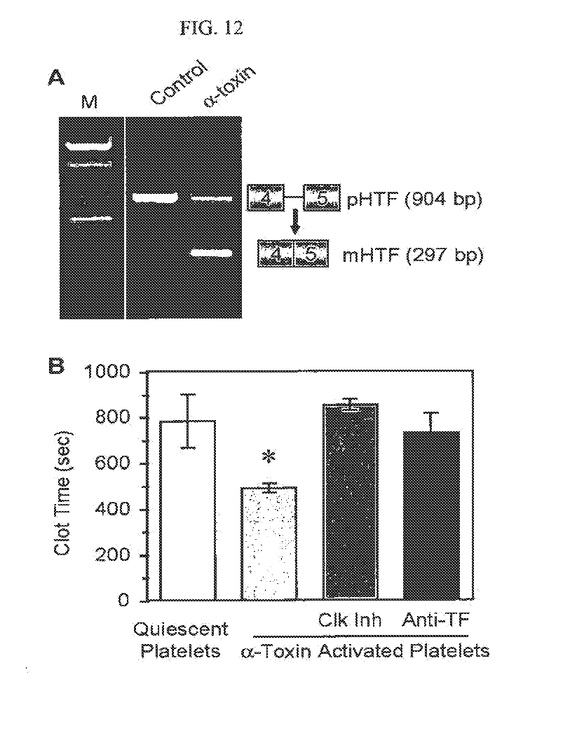

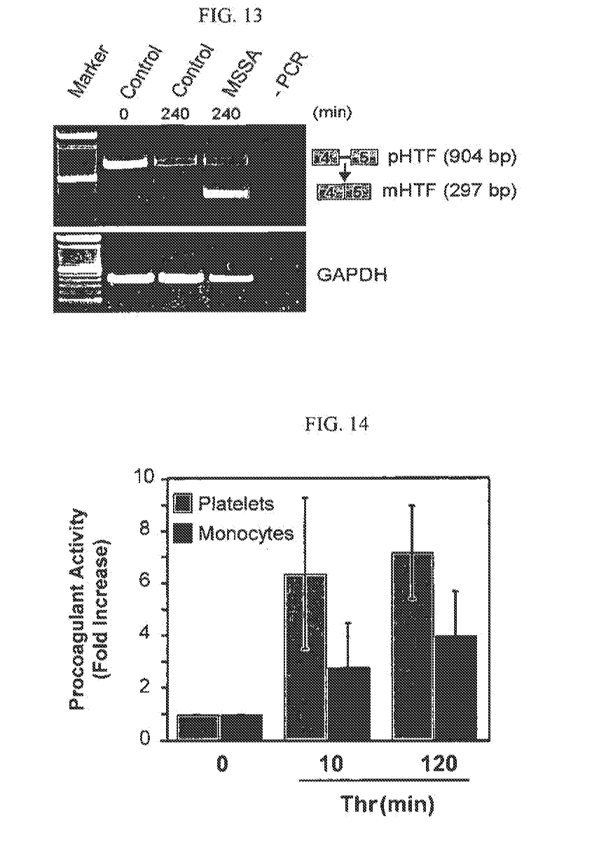

Next it was determined if bacterial products induce TF pre-mRNA splicing in platelets. E. coli, LPS, S. aureus or α-toxin induced TF pre-mRNA splicing in platelets from healthy volunteers (FIGS. 11, 12, and 13). Both agonists also triggered generation of procoagulant activity, consistent with accelerated plasma clot formation (FIGS. 11 and 12). Similar to activation with thrombin (FIG. 4), splicing-mediated responses induced by bacterial products were blocked by Clk1 inhibition (see, FIGS. 11 and 12).

Platelets from healthy subjects were left quiescent or activated with α-toxin (10 ng/ml; List Biological Laboratories Inc.) or lipopolysaccharide (LPS; 100 ng/ml; Sigma), toxins produced by gram-positive Staph. aureus and gram-negative E. Coli, respectively. Platelets were kept quiescent or activated in suspension with MRSA or E. coli at a bacteria to platelet ratio of 1:10. The bacteria were obtained from blood cultures from sepsis patients by ARUP, a national reference laboratory under standard conditions. For these studies, the bacteria were swiped from the culture plate, re-suspended, and the desired concentration was adjusted against a standard solution using a colorimeter (Vitek Colorimeter, bioMéreux, Inc.).

Septic patients with E. coli or S. aureus bacteremia were identified and after isolation, each bacterial strain was incubated with washed platelets and TF pre-mRNA splicing was examined. As shown in FIGS. 11 and 12, E. coli and S. aureus induced rapid TF pre-mRNA splicing in platelets.

Together, these data demonstrate that products from both gram-negative and gram-positive bacteria produce agonists that can activate pre-mRNA splicing and TF synthesis via a Clk1-dependent mechanism.

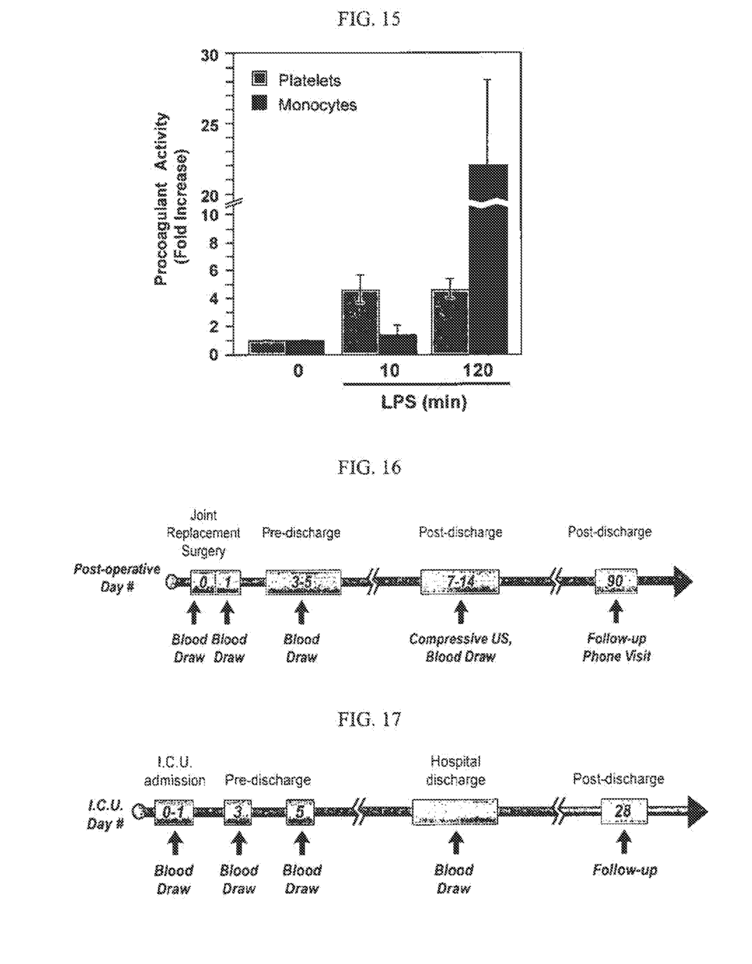

Since LPS is a powerful agonist for synthesis of TF by monocytes, TF-dependent procoagulant activity in monocyte cells that were isolated from the same donor were measured. Platelets and monocytes were stimulated with LPS and, for comparison, thrombin. Thrombin-stimulated monocytes generated less procoagulant activity than platelets (FIG. 14). In contrast, LPS-stimulated monocytes generated significant amounts of procoagulant activity after 120 minutes (FIG. 15), consistent with de novo transcription and processing of TF mRNA. However, LPS did not induce TF activity in monocytes after 10 minutes whereas it was present in LPS-activated platelets at the same time point (FIG. 15). This indicates that TF expression in monocytes has temporal and agonist-specific features. The results also suggest that depending on the time point and the agonist, platelets were as robust a source of TF activity as were monocytes.

MRSA, E. coli, α-toxin or lipopolysaccheride (LPS) induced TF pre-mRNA splicing in platelets isolated from healthy subjects. Membranes isolated from α-toxin and LPS stimulated platelets, but not unstimulated platelets, also accelerated clotting of human plasma. This response was blocked when the platelets were pre-incubated with the splicing inhibitor Tg003 or when the isolated membranes from α-toxin or LPS stimulated platelets were pre-incubated with a TF neutralizing antibody prior to being placed in the plasma.

The demonstration that agonists prevalent in sepsis induce TF pre-mRNA splicing in platelets from healthy donors indicates that similar mRNA expression patterns are likely to prevail in platelets from sepsis patients. For example, within 24 hours of ICU admission, platelets from over half of the enrolled septic patients express spliced TF mRNA (FIG. 1A), in sharp contrast, platelets from healthy subjects express unspliced, TF pre-mRNA. The incidence of pre-mRNA splicing increased as APACHE II scores rose (FIG. 1B) and that higher APACHE II scores were associated with increased mortality. The mortality rate also increased (2.6 fold) in septic patients whose platelets expressed spliced TF mRNA. Consistent with these findings, patients who spliced TF pre-mRNA were less likely to survive their sepsis.

### Example IV

In order to confirm and further delineate the correlation between TF pre-mRNA splicing in the elderly and prediction, diagnosis or prognosis of coagulation disorders, such as VTE, healthy elderly volunteers aged 65 and older who consent to participate are eligible for inclusion in a further study. Subjects are excluded from the study if they are unable or unwilling to give consent, are an active smoker, have had an infection in the last 14 days, have had a previous platelet transfusion or blood transfusion within the past 4 months, have thrombocytopenia (platelet count <50,000×106/L), have active malignancy, have a history of VTE, or have cardiovascular disease, diabetes, COPD, or heart failure. In addition, subjects who are taking prescribed medications or aspirin may be excluded.

Healthy young volunteers between the ages of 18-40 who consent to participate are eligible for inclusion. The same exclusion criteria described for the healthy elderly volunteers are used here.

Whole blood is collected from each subject and a small fraction of it is used (e.g., sent to a National Reference laboratory) to obtain platelet and leukocyte counts as well as a coagulation profile (i.e., fibrinogen, anti-thrombin III, protein C and S, quantitative D-dimers, and prothrombin and partial thromboplastin times). The remaining blood is used to purify platelets and possibly monocytes^{18,20,42,56}.

Whole blood from each subject is preferably delivered to the laboratory within 15-20 minutes of collection. The sample may be labeled with a unique identifier to protect the anonymity of the subject. Platelets, microparticles, platelets and microparticles, monocytes, and/or leukocytes are isolated from the same blood sample and sterile conditions are used throughout the isolation procedure to ensure that the isolated cells are not exposed to bacterial products in the laboratory^{81,19,40-42,56}.

Pre-mRNA splicing and protein synthesis is measured in platelets from elderly and young subjects. In parallel, expression of IL-1β and TF mRNA and protein in monocytes may be monitored. This will allow for comparison of pre-mRNA splicing between platelets and monocytes in each subject or population group.

In addition, signal-dependent pre-mRNA splicing and protein synthesis is measured in platelets from elderly and young subjects. In parallel, expression of IL-1β and TF mRNA and protein in monocytes may be monitored. This will allow for comparison of synthetic responses between activated platelets and monocytes in each subject or population group.

A fraction of the freshly-isolated platelets and possibly monocytes may be immediately processed to obtain a baseline mRNA expression and corresponding protein profile for each cell population. The remaining platelets and possibly monocytes are stimulated separately with thrombin (0.1 U/ml). Platelets and monocytes may be stimulated for 1 hour—a time point where pre-mRNA splicing is nearly complete and protein accumulation is evident¹²⁻¹⁴. However, in donors from whom sufficient cell numbers are retrieved, a more thorough time course (e.g., 0.5, 1, 2 and 4 hours) and concentration-dependent responses to thrombin (e.g., 0.1, 0.5 and 1.0 U/ml) may be evaluated. In addition to thrombin, monocytes may be stimulated with lipopolysaccharide (LPS) for 4 hours, a condition that can serve as a positive control for IL-1β and TF synthesis in this nucleated cell.

A portion of the cellular pellet may be used to isolate total RNA using known procedures²⁰. The RNA may be treated with DNase to remove trace amounts of genomic DNA. Platelet RNA may be amplified (MessageAmp™ II a RNA amplification, Ambion) because transcript levels from platelets are approximately 100-fold less abundant than monocyte-derived RNA. Amplification of platelet RNA allows characterization of mRNA expression patterns and corresponding protein profiles from the same sample. Using amplification procedures, it is possible to generate approximately 2-3 μg of RNA from 1×10⁸ total platelets (˜0.5-1 ml whole blood assuming a count of 200,000 platelets/μl).

The amplified RNA is used to screen for differential expression patterns (i.e., spliced or unspliced) between platelets isolated from the elderly and young. mRNAs may be considered unspliced if only a pre-mRNA band is detected. The mRNAs may be considered spliced if a processed mRNA species, in the presence or absence of a pre-mRNA band, is detected. Optionally, the degree of splicing may be measured, however, because this analysis is semi-quantitative this step may be omitted. Detection of pre-mRNA and spliced mRNA for IL-1β and TF is determined using exon spanning primer sets^{19,20}. The same primer sets may be used to screen for transcribed IL-1β and TF mRNA in monocytes by RNase protection and real-time PCR. This allows the comparison of transcribed mRNA expression levels between the elderly and young. Spliced mRNA for IL-1β, but not its pre-mRNA, has been detected in LPS-stimulated monocytes suggesting that splicing is a co-transcriptional event in monocytes¹². mRNA expression patterns in both cells may be normalized to an internal control, such as GAPDH. Optionally, α_{IIb} and CD45 mRNA may be screened, using α_{IIb} as a platelet specific marker and CD45 as a leukocyte specific marker. Screens for these two transcripts may be used to ensure that the platelet preparations are devoid of leukocytes and conversely, the leukocyte preparations do not contain platelets.

TF-dependent pro-coagulant activity is measured in platelets, monocytes, and/or microparticles²⁰. TF antigen expression may be measured by ELISA in the microparticle-free supernatants of stimulated platelets and monocytes. Soluble TF antigen levels may also be determined in plasma samples from the elderly and young to determine if it correlates with pre-mRNA splicing or mRNA transcription patterns in freshly-isolated platelets or monocytes, respectively. Platelets or monocytes from elderly subjects that have altered TF-dependent procoagulant activity compared to young volunteers may also be measured to determine clotting times²⁰. For such studies, the platelets, monocytes or microparticles may be incubated with pooled human plasma and clotting times determined by any method known in the art²⁰.

Because thrombin induces pre-mRNA splicing and associated protein synthetic responses in platelets isolated from young subjects, it is expected that thrombin will induce pre-mRNA splicing and associated protein synthetic responses in platelets isolated from elderly subjects. Likewise, the elderly are expected to express Clk1 protein and redistribute it into focal contact points. Optionally, elderly subjects may be tested for activation by thrombin and activation of Clk1. For the redistribution assays, platelets may be activated with thrombin as they adhere to immobilized fibrinogen or collagen²⁰.

An immune complex kinase assay specific for Clk1 enzymatic activity in platelets may be performed by methods known in the art²⁰. These assays may be done in the presence of Tg003, a benzothiazole derivative that inhibits Clk1 activity, or its structurally inactive analogous compound Tg009²⁰. Alternatively, other Clk1 inhibitors may be used. However, Tg003 was recently demonstrated to be a specific Clk1 inhibitor that does not alter platelet adhesion, spreading, and aggregation²⁰.

Once Clk1 activity is characterized, it may be desirable to confirm that this enzymatic pathway controls pre-mRNA splicing events in platelets from the elderly. It is believed that Tg003 will prevent platelets from accelerating the rate of plasma clot formation in elderly subjects.

### Example V

Increased age is a risk factor for deep vein thrombosis (DVT) following total joint replacement, and there is considerable debate as to whether anti-platelet treatment is beneficial in preventing DVT⁵⁸⁻⁶¹. However, the argument against a role for platelets in DVT does not take into account TF pre-mRNA splicing in this anucleate cell. In addition, the present data indicates that neither aspirin nor heparin block pre-mRNA splicing in platelets (see, FIG. 17). Thus, platelets possess biological activities important in the pathogenesis of DVT; activities which are possibly increased in the elderly and which are not blocked by drugs used clinically to prevent DVT.

Nearly 25% of otherwise relatively healthy elderly patients who undergo elective knee or hip replacement will develop symptomatic or asymptomatic post-operative DVT. This occurs despite the use of currently available VTE prophylaxis treatments, which are not expected to prevent many of the functions of platelets, including production of TF-dependent coagulation activity through TF pre-mRNA splicing.

Patterns of post-transcriptional gene expression, e.g., TF pre-mRNA splicing patterns, in platelets are believed to prospectively identify elderly patients having a higher risk of developing DVT after joint replacement surgery.

Patients >65 years of age scheduled for elective hip or knee replacement are enrolled for the purpose of confirming a correlation between TF pre-mRNA splicing or TF-dependent coagulation activity and DVT/VTE detected by ultrasound imaging and clinical evaluation. Although the knee and hip surgery patients tend to develop distal (i.e., calf vein) and proximal (i.e., thigh vein) DVT, respectively, the study described herein is designed to detect both proximal and distal DVT.

Patients (>65 years) scheduled for elective primary hip or knee replacement and able to consent are eligible for inclusion. Patients may be enrolled regardless of which medications they are taking based on the data that prescribed medications or aspirin have no effect on the gene expression pathways to be tested. Patients are excluded from the study if they are unable or unwilling to give consent, are undergoing revision arthroplasty (because of the significant risk of having sustained unrecognized DVT during the initial procedure), have had a previous platelet transfusion or blood transfusion within the past 4 months, have severe thrombocytopenia (platelet count <50,000×106/L), have active malignancy, or have a history of VTE. Patients with a history of known, documented thrombophilia (Factor V Leiden, Prothrombin 20210 gene mutation, deficiencies of AT III, protein C or S, or homocysteinemia) are also excluded.

Following informed consent, blood is drawn (˜20-40 ml) pre-operatively, on post-operative day (POD) 1 (see FIG. 16 depicting a basic protocol) and within 24 hours of discharge from the hospital for various assays. At POD 7-14, another blood sample is collected in parallel with a bilateral, compression ultrasound imaging examination specifically focused on detection of DVT in the femoral, popliteal or calf vein distributions. All of the patients receive a follow-up phone call at approximately POD 90 and are asked a set of questions to screen for possible symptoms of DVT or pulmonary embolism (PE). Potential episodes of VTE are confirmed by ultrasound imaging.

DVT or clinical VTE are diagnosed upon: (1) a single ultrasound detection of a non-compressible segment of a proximal deep vein (femoral or popliteal) in either lower extremity; or the repeated detection of a non-compressible segment of the same calf vein 3-5 days later (including the gastrocnemius and soleal veins), or extension into a larger, more proximal vein; or (2) symptomatic PE diagnosed by computed tomography, catheter angiography, or high probability ventilation-perfusion scan.

Whenever possible, the finding of a non-compressible segment on ultrasound is corroborated by the presence of a filling defect on color imaging and the presence of intraluminal material on gray-scale imaging. However, the finding of a non-compressible segment by itself is sufficient for the diagnosis of DVT^{62,63}.

All patients are to receive clinically indicated imaging evaluation for possible symptomatic DVT or PE at the discretion of their primary physician. Thus, the DVT endpoint can be reached as a result of either a study ordered for appropriate clinical indications or based on the findings of the examinations in otherwise asymptomatic patients.

Screening for DVT at this post-discharge time point is based on the emerging appreciation that the majority of post-arthroplasty DVT is first detected after the patient leaves the hospital. This is in part due to current clinical practice, in which patients are being discharged at earlier times after surgery. An asymptomatic DVT that is undetected prior to discharge may still be present and detected at the first post-discharge visit (POD 7-14). Schindler et al.⁶⁴ did not see evidence of thrombus resolution in asymptomatic DVT for at least 4-6 weeks after joint replacement. More rigorous analyses of DVT resolution documented that the interquartile range of spontaneous lysis in the posterior tibial vein was 28-182 days with all other lower extremity veins having longer times to thrombus resolution.

Ultrasound imaging has supplanted ascending venography as the gold standard for detecting DVT since it does not carry the associated risks of allergic reactions to contrast, nephropathy or even provoking a DVT when one was not present before the test^{62,63}. Protocols that utilize closely spaced compressions at multiple points in three regions of the lower extremity (thigh, popliteal fossa, calf) combined with color imaging in anatomic regions difficult to compress (the adductor canal) are highly reliable with sensitivities exceeding 90% for proximal DVT, even in asymptomatic patients. While the diagnostic sensitivity is lower for calf DVT, use of screening ultrasound imaging still capable of identifying DVT in 20-30% of post-arthroplasty patients. Therefore, considering the risks associated with venography, ultrasound imaging is preferable detection method in many situations.

For each of the four blood draws outlined in FIG. 17 a CBC and coagulation profile may be obtained. The remaining blood is used to isolate platelets, monocytes and/or microparticles. Plasma may be used for alternative biomarker analyses. During cell isolation, the lymphocyte fraction is stored so that DNA analyses for molecular thrombophilia can be performed if warranted. TF pre-mRNA expression patterns and TF-dependent procoagulant activity are assayed in platelets and microparticles.

Currently used medications, past medical history, smoking status, hip or knee replacement, type of anesthesia, estimated blood loss and replacement, post-operative complications, time to ambulation, length of stay, and/or discharge disposition may be recorded for each subject.

It is believed that patients whose platelets express spliced TF mRNA pre-operatively, or whose platelets splice pre-mRNA into mature mRNA in response to joint replacement surgery, have a higher risk of developing DVT in the post-operative period than those who do not.

Optionally, a cohort of young patients who receive joint replacement surgery are also measured. In addition, a pre-operative ultrasound may be performed to screen for patients with undiagnosed VTE, however, the probability of detecting a DVT at this time is low and these tests are not typically included in studies of VTE after joint replacement surgery.

Since the presence of DVT is a dichotomous variable as is the presence or absence of pre-mRNA splicing, a Chi-square test may be used to assess statistical significance.

### Example VI

In order to confirm and further delineate the correlation between TF pre-mRNA splicing and diagnosis, prognosis or disease prediction for sepsis, patients meeting the criteria for SIRS (systemic inflammatory response syndrome) and cable of providing informed consent are enrolled for study. Patients meet the criteria for SIRS if they have two of the following criteria: (1) temperature <36° C. or >38° C., (2) heart rate >90 beats per minute, (3) respiratory rate >20 breaths per minute or PaCO2<32 mm Hg, (4) white blood cell count ≧12,000 or ≦4,000 cells/mm3 or >10% bands, as outlined in published consensus statements'. In addition, patients must have an identified focus of infection (thus meeting consensus criteria for sepsis⁵⁷) including abdominal, lung, or urinary tract infection with or without bacteremia. Bacteremia is documented by blood cultures. Pneumonia is defined as the presence of a new infiltrate on a chest x-ray or chest computerized tomography (CT). Urinary tract infection is defined as evidence of bacteriuria, white blood cells, positive leukocyte esterase or nitrates on a clean catch urine sample or bacteria isolated from urine cultures. Patients receiving prophylactic heparin, which is indicated in the absence of active or extreme risk of bleeding³, are also included. In addition, no exclusion is based on medication, because commonly used medications such as aspirin, other anti-platelet drugs, or warfarin do not affect pre-mRNA splicing events in platelets.

Patients are excluded if they are unable or unwilling to give consent and no family member is available to provide consent, have had a platelet transfusion within the past 14 days, or have had a blood transfusion within the past 4 months.

Patients admitted with a diagnosis of acute, decompensated heart failure (or heart failure exacerbation) are admitted in order to examine a group of acutely ill subjects with systemic manifestations in parallel with the analysis of samples from patients with sepsis. Patients with acute, decompensated heart failure have circulating cytokine profiles that are similar to those in patients with sepsis, including IL-6, and thus represent a relevant, non-infected control group. These patients are age- and gender-frequency matched to the patients with sepsis.

Patients are excluded if they are unable or unwilling to give informed consent, have had a platelet transfusion within the past 14 days, have had a blood transfusion within the past 4 months, meet consensus criteria for sepsis, have had an infection within the past 14 days, have received thrombolytic therapy within the past 7 days, are pregnant, or have DIC.

Non-medicated, healthy volunteers who agree to participate are enrolled. These patients are age- and gender-frequency matched to the patients with sepsis.

Patients are excluded from the study if they are unable or unwilling to give consent, have had an infection over the last 14 days, have had a platelet transfusion within the past 14 days or blood transfusion within the past 4 months.

Screening for enrollment of septic patients into this trial is preferably done in the ICU of a hospital. The frequency matching of controls to septic patients provides balance on sex and a broad age category (<65 or >65 years). The two controls groups, hospitalized and non-hospitalized, are selected to achieve balance with the sepsis group on the four possible sex and age category combinations, so that the proportion of control patients in each category matches the sepsis group.

Following informed consent, serial blood samples (ICU day 0-1, 3, 5 and within 24 hours of hospital discharge in survivors) are collected (˜25-30 ml) from septic patients after careful consideration of the patient's clinical status (see, FIG. 17). The initial sample (e.g., day 0-1) is drawn within 24 hours of the diagnosis of sepsis. The blood samples are preferably coded immediately to preserve patient confidentiality and delivered to a laboratory for processing. Cell counts, leukocyte differentials and coagulation indices are preferably assessed in parallel. Clinical data requisite for documentation of septic syndromes, medications, laboratory data, demographics, and clinical outcomes may also be collected. Patient mortality is assessed throughout the study and at 28 days after admission to the ICU.

For the hospitalized control group, a blood sample is preferably obtained within 24 hours of admission to the hospital and within 24 hours of discharge. For the healthy volunteers, one blood sample may be obtained. Coagulation indices and relevant clinical data may be collected at the time of each blood draw.

Protein C and protein S levels are decreased in sepsis and correlate with mortality risk. Therefore, plasma fibrinogen levels, quantitative D-dimers, prothrombin time and activated partial thromboplastin times, protein C and protein S levels, and/or anti-thrombin III levels may be measured to determine if they correlate with the incidence of pre-mRNA splicing in platelets in septic patients. These indices of coagulation may be measured in whole blood obtained from healthy controls, hospitalized patients without sepsis, and ICU patients with sepsis.

This study is designed to investigate pre-mRNA splicing in platelets isolated from septic patients as compared to age- and sex-matched controls and hospitalized, non-septic patients. The primary study endpoint is preferably 28-day mortality and the incidence of pre-mRNA splicing and corresponding protein responses are anticipated to be increased in patients who do not survive. Based on the initial data, it is anticipate that the incidence of IL-1β and/or TF pre-mRNA splicing in platelets will be increased in patients with sepsis versus the control cohort groups. In addition, altered pre-mRNA splicing and corresponding protein responses are anticipated to correlate with the severity of illness (e.g., APACHE II, SOFA, and SAPS scores) and abnormal coagulation indices (e.g., quantitative D-dimers, fibrinogen levels, anti-thrombin III, protein C, protein S, prothrombin and activated partial thromboplastin times). Optionally, it is also possible to determine the correlation between pre-mRNA splicing and patients with documented bacteremia.

### Example VII

Bacteria may be isolated from septic patients with blood cultures positive for either E. coli or S. aureus (BACTEC 9240, Bectin Dickenson) and subcultured. Pure culture isolates may be stored in glycerol stock solution at −70° C. Isolation, phenotypic identification by classical methods, genotypic identification by 16S rRNA gene sequencing (when indicated), and subculturing of the bacteria may be conducted using methodologies well known in the art. For example, the isolates may be grown at 37° C. to log phase (e.g., 18 hrs, 37° C.) in antibiotic-free brain heart infusion (BHI) broth to circumvent any potential antibiotic-mediated influences on bacterium-platelet interactions. E. coli and S. aureus bacterial strains may be counted (cfu) and incubated with isolated cells or whole blood.

Cell isolation procedures are well known in the art. Importantly, these procedures have been rigorously validated, reviewed and yield platelet and monocyte preparations that are devoid of contaminating cell types and microbial products.

A portion of the cellular pellet may be used to isolate total RNA. The RNA is preferably treated with DNase to remove trace amounts of genomic DNA²⁰ and intact mRNA is isolated from the total RNA preparation (Dynabeads® Oligo(dT)25, Dynal Biotech). The platelet RNA is preferably amplified (MessageAmp™ II aRNA amplification, Ambion) to generate enough template for the desired analyses.

The amplified RNA may be used to screen for differential expression patterns (i.e., spliced and unspliced) between septic patients and control cohorts. For initial analysis, mRNAs may be considered unspliced if only a pre-mRNA band is detected and considered spliced if a processed mRNA species is detected, in the presence or absence of a pre-mRNA product. Alternatively, the spliced mRNA products may be sub-categorized into partially spliced (PS; presence of both pre-mRNA and spliced mRNA species) or completely spliced (CS; presence of only a mature mRNA species) (also see FIG. 8). Detection of pre-mRNA and spliced mRNA for IL-1β and TF may be determined using exon spanning primer sets^{19,20}. Optionally, α_{IIb} and CD45 mRNA may be screened for, using α_{IIb} as a platelet specific marker and CD45 as a leukocyte specific marker. Screens for these two transcripts may be used to ensure that the platelet preparations are devoid of leukocytes, and/or conversely, that the leukocyte preparations are devoid of platelets. All of the mRNA expression studies may be normalized to an internal control, such as GAPDH.

TF-dependent procoagulant activity may be measured as previously described in platelets, monocytes, and microparticles²⁰. TF antigen expression may be measured by ELISA in the plasma or cell-free supernatants and correlated with TF pre-mRNA splicing. TF-dependent clotting may be measured by methods known in the art²⁰.

### Example VIII

In order to further delineate the correlation between TF pre-mRNA splicing and the diagnosis, prognosis or disease prediction for sepsis, patients that may be predisposed to sepsis and who are capable of providing informed consent are enrolled for study. Control patients may also be enrolled

Pre-mRNA splicing in platelets isolated from subjects that may be in the early stages of sepsis and that are eventually diagnosed with sepsis are compared to subjects that are not eventually diagnosed with sepsis. The primary study endpoint is either diagnosis with sepsis and/or remission of all criteria indicative of possible sepsis. In subjects ultimately diagnosed with sepsis the 28-day mortality rate may also be monitored and/or used as an additional study endpoint. Blood samples from subjects are measured for pre-mRNA splicing, either directly or indirectly, and the level of TF pre-mRNA splicing is plotted against disease progression and diagnosis. Based on initial data, it is anticipate that the incidence of IL-1β and/or TF pre-mRNA splicing in platelets will be increased in patients eventually diagnosed with sepsis. In addition, elevated levels of TF pre-mRNA splicing are believed to correlate with an increased probability of an eventual diagnosis of sepsis. Therefore, an elevated level of TF pre-mRNA splicing is believed to be predictive of sepsis.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein, including:
1. Cohen J. The immunopathogenesis of sepsis. Nature. 2002; 420:885-891.
2. Zimmerman G A, Albertine, K. H., McIntyre, T. M. Pathogenesis of Sepsis and Septic-Induced Lung Injury. In: Matthay M A, ed. Acute Respiratory Distress Syndrome. Vol. 179. New York: Marcel Dekker, Inc.; 2003:245-287.
3. Angus D C, Linde-Zwirble W T, Lidicker J, Clermont G, Carcillo J, Pinsky M R. Epidemiology of severe sepsis in the United States: analysis of incidence, outcome, and associated costs of care. Crit Care Med. 2001; 29:1303-1310.
4. Levi M. Platelets at a crossroad of pathogenic pathways in sepsis. J Thromb Haemost. 2004; 2:2094-2095.
5. Vincent J L, Yagushi A, Pradier O. Platelet function in sepsis. Crit. Care Med. 2002; 30:5313-317.
6. Schwertz H, Weyrich, A. S., Zimmerman, G. A. Cellular interactions of platelets, leukocytes, and endothelium in systemic inflammatory responses and sepsis. In: Castro Faria Neto H C, David, C. M., ed. Sepsis: From Bench to Bedside. Rio de Janerio: Revinter Press; 2007: In press.
7. Stephan F, Hollande J, Richard O, Cheffi A, Maier-Redelsperger M, Flahault A. Thrombocytopenia in a surgical ICU. Chest. 1999; 115:1363-1370.
8. Strauss R, Wehler M, Mehler K, Kreutzer D, Koebnick C, Hahn E G. Thrombocytopenia in patients in the medical intensive care unit: bleeding prevalence, transfusion requirements, and outcome. Crit Care Med. 2002; 30:1765-1771.
9. Vanderschueren S, De Weerdt A, Malbrain M, Vankersschaever D, Frans E, Wilmer A, Bobbaers H. Thrombocytopenia and prognosis in intensive care. Crit Care Med. 2000; 28:1871-1876.
10. Warkentin T E, Aird W C, Rand J H. Platelet-endothelial interactions: sepsis, HIT, and antiphospholipid syndrome. Hematology (Am Soc Hematol Educ Program). 2003:497-519.
11. Esmon C T, Fukudome K, Mather T, Bode W, Regan L M, Stearns-Kurosawa D J, Kurosawa S. Inflammation, sepsis, and coagulation. Haematologica. 1999; 84:254-259.
12. Weyrich A S, Zimmerman G A. Platelets: signaling cells in the immune continuum. Trends Immunol. 2004; 25:489-495.
13. Evans G, Lewis A F, Mustard J F. The role of platelet aggregation in the development of endotoxin shock. Br J Surg. 1969; 56:624.
14. Cowan D H, Bowman L S, Fratianne R B, Ahmed F. Platelet aggregation as a sign of septicemia in thermal injury. A prospective study. Jama. 1976; 235:1230-1234.
15. Yaguchi A, Lobo F L, Vincent J L, Pradier O. Platelet function in sepsis. J Thromb Haemost. 2004; 2:2096-2102.
16. Alt E, Amann-Vesti B R, Madl C, Funk G, Koppensteiner R. Platelet aggregation and blood rheology in severe sepsis/septic shock: relation to the Sepsis-related Organ Failure Assessment (SOFA) score. Clin Hemorheol Microcirc. 2004; 30:107-115.
17. Gawaz M, Dickfeld T, Bogner C, Fateh-Moghadam S, Neumann F J. Platelet function in septic multiple organ dysfunction syndrome. Intensive Care Med. 1997; 23:379-385.
18. Denis M M, Tolley N D, Bunting M, Schwertz H, Jiang H, Lindemann S, Yost C C, Rubner F J, Albertine K H, Swoboda K J, Fratto C M, Tolley E, Kraiss L W, McIntyre T M, Zimmerman G A, Weyrich A S. Escaping the nuclear confines: signal-dependent pre-mRNA splicing in anucleate platelets. Cell. 2005; 122:379-391.
19. Lindemann S, Tolley N D, Dixon D A, McIntyre T M, Prescott S M, Zimmerman G A, Weyrich A S. Activated platelets mediate inflammatory signaling by regulated interleukin 1beta synthesis. J Cell Biol. 2001; 154:485-490.
20. Schwertz H, Tolley N D, Foulks J M, Denis M M, Risenmay B W, Buerke M, Tilley R E, Rondina M T, Harris E M, Kraiss L W, Mackman N, Zimmerman G A, Weyrich A S. Signal-dependent splicing of tissue factor pre-mRNA modulates the thrombogenecity of human platelets. J Exp Med. 2006; 203:2433-2440.
21. Brogren H, Karlsson L, Andersson M, Wang L, Erlinge D, Jern S. Platelets synthesize large amounts of active plasminogen activator inhibitor 1. Blood. 2004; 104:3943-3948.
22. Lindemann S, Tolley N D, Eyre J R, Kraiss L W, Mahoney T M, Weyrich A S. Integrins regulate the intracellular distribution of eukaryotic initiation factor 4E in platelets. A checkpoint for translational control. J Biol Chem. 2001; 276:33947-33951.
23. Pabla R, Weyrich A S, Dixon D A, Bray P F, McIntyre T M, Prescott S M, Zimmerman G A. Integrin-dependent control of translation: engagement of integrin alphaIIbbeta3 regulates synthesis of proteins in activated human platelets. J Cell Biol. 1999; 144:175-184.
24. Weyrich A S, Dixon D A, Pabla R, Elstad M R, McIntyre T M, Prescott S M,
Zimmerman G A. Signal-dependent translation of a regulatory protein, Bcl-3, in activated human platelets. Proc Natl Acad Sci USA. 1998; 95:5556-5561.
25. Weyrich A S, Lindemann, S., Tolley, N. D., Kraiss, L. W., Dixon, D. A., Mahoney, T. M., Prescott, S. M., McIntyre, T. M., Zimmerman, G. A. Change in protein phenotype without a nucleus: translational control in platelets. Seminars in Thrombosis and Hemostasis. 2004; 30:493-500.
26. Lindemann S, McIntyre, T M, Prescott, S M, Zimmerman, G A, and Weyrich, A S. Platelet Signal-Dependent Protein Synthesis. In: Fitzgerald DJaQ, M., ed. Platelet Function Assessment, Diagnosis, and Treatment. Totowa: The Humana Press Inc.; 2005:151-176.
27. Evangelista V, Manarini S, Di Santo A, Capone M L, Ricciotti E, Di Francesco L, Tacconelli S, Sacchetti A, D'Angelo S, Scilimati A, Sciulli M G, Patrignani P. De novo synthesis of cyclooxygenase-1 counteracts the suppression of platelet thromboxane biosynthesis by aspirin. Circ Res. 2006; 98:593-595.
28. Weyrich A S, Denis M M, Schwertz H, Tolley N D, Foulks J, Spencer E, Kraiss L W, Albertine K H, McIntyre T M, Zimmerman G A. mTOR-dependent synthesis of Bcl-3 controls the refraction of fibrin clots by activated human platelets. Blood. 2006.
29. Gando S, Kameue T, Morimoto Y, Matsuda N, Hayakawa M, Kemmotsu O. Tissue factor production not balanced by tissue factor pathway inhibitor in sepsis promotes poor prognosis. Crit Care Med. 2002; 30:1729-1734.
30. Drake T A, Morrissey J H, Edgington T S. Selective cellular expression of tissue factor in human tissues. Implications for disorders of hemostasis and thrombosis. Am J Pathol. 1989; 134:1087-1097.
31. Mackman N. Role of tissue factor in hemostasis and thrombosis. Blood Cells Mol Dis. 2006; 36:104-107.
32. Drake T A, Cheng J, Chang A, Taylor F B, Jr. Expression of tissue factor, thrombomodulin, and E-selectin in baboons with lethal Escherichia coli sepsis. Am J Pathol. 1993; 142:1458-1470.
33. Johnson M, Ramey E, Ramwell P W. Sex and age differences in human platelet aggregation. Nature. 1975; 253:355-357.
34. Meade T W, Vickers M V, Thompson S G, Stirling Y, Haines A P, Miller G J. Epidemiological characteristics of platelet aggregability. Br Med J (Clin Res Ed). 1985; 290:428-432.
35. Nordoy A, Svensson B, Haycraft D, Hoak J C, Wiebe D. The influence of age, sex, and the use of oral contraceptives on the inhibitory effects of endothelial cells and PGI2 (prostacyclin) on platelet function. Scand J Haematol. 1978; 21:177-187.
36. Vericel E, Croset M, Sedivy P, Courpron P, Dechavanne M, Lagarde M. Platelets and aging. I—Aggregation, arachidonate metabolism and antioxidant status. Thromb Res. 1988; 49:331-342.
37. Emery J D, Leifer D W, Moura G L, Southern P, Morrissey J H, Lawrence J B. Whole-blood platelet aggregation predicts in vitro and in vivo primary hemostatic function in the elderly. Arterioscler Thromb Vasc Biol. 1995; 15:748-753.
38. Macpherson C R, Jacobs P. Bleeding time decreases with age. Arch Pathol Lab Med. 1987; 111:328-329.
39. Brown M D, Hogikyan R V, Dengel D R, Supiano M A. Sodium-sensitive hypertension is not associated with higher sympathetic nervous system activity in older hypertensive humans. Am J Hypertens. 2000; 13:873-883.
40. Lee C C, Sidani M A, Hogikyan R V, Supiano M A. The effects of ramipril on sympathetic nervous system function in older patients with hypertension. Clin Pharmacol Ther. 1999; 65:420-427.
41. Supiano M A, Hogikyan R V. High affinity platelet alpha 2-adrenergic receptor density is decreased in older humans. J Gerontol. 1993; 48:B173-179.
42. Supiano M A, Hogikyan R V, Sidani M A, Galecki A T, Krueger J L. Sympathetic nervous system activity and alpha-adrenergic responsiveness in older hypertensive humans. Am J. Physiol. 1999; 276:E519-528.
43. Supiano M A, Linares O A, Halter J B, Reno K M, Rosen S G. Functional uncoupling of the platelet alpha 2-adrenergic receptor-adenylate cyclase complex in the elderly. J Clin Endocrinol Metab. 1987; 64:1160-1164.
44. Supiano M A, Neubig R R, Linares O A, Halter J B, Rosen S G. Effects of low-sodium diet on regulation of platelet alpha 2-adrenergic receptors in young and elderly humans. Am J Physiol. 1989; 256:E339-344.
45. Merli G J. Pathophysiology of venous thrombosis, thrombophilia, and the diagnosis of deep vein thrombosis-pulmonary embolism in the elderly. Clin Geriatr Med. 2006; 22:75-92, viii-ix.
46. Jaffer A K, Brotman D J. Prevention of venous thromboembolism in the geriatric patient. Clin Geriatr Med. 2006; 22:93-111, ix.
47. Anderson F A, Jr., Wheeler H B, Goldberg R J, Hosmer D W, Patwardhan N A, Jovanovic B, Forcier A, Dalen J E. A population-based perspective of the hospital incidence and case-fatality rates of deep vein thrombosis and pulmonary embolism. The Worcester DVT Study. Arch Intern Med. 1991; 151:933-938.
48. Longo M G, Greco A, Pacilli M, D'Ambrosio L P, Scarcelli C, Grandone E, Fanelli R, Di Minno G, Pilotto A. Deep venous thrombosis in elderly hospitalized patients: prevalence and clinical features. Aging Clin Exp Res. 2005; 17:42-45.
49. Ho K M, Dobb G J, Knuiman M, Finn J, Lee K Y, Webb S A. A comparison of admission and worst 24-hour Acute Physiology and Chronic Health Evaluation II scores in predicting hospital mortality: a retrospective cohort study. Crit. Care. 2006; 10:R4.
50. Le Gall J R, Lemeshow S, Saulnier F. A new Simplified Acute Physiology Score (SAPS II) based on a European/North American multicenter study. Jama. 1993; 270:2957-2963.
51. Oda S, Hirasawa H, Sugai T, Shiga H, Nakanishi K, Kitamura N, Sadahiro T, Hirano T. Comparison of Sepsis-related Organ Failure Assessment (SOFA) score and CIS (cellular injury score) for scoring of severity for patients with multiple organ dysfunction syndrome (MODS). Intensive Care Med. 2000; 26:1786-1793.
52. Healy A M, Pickard M D, Pradhan A D, Wang Y, Chen Z, Croce K, Sakuma M, Shi C, Zago A C, Garasic J, Damokosh A I, Dowie T L, Poisson L, Lillie J, Libby P, Ridker P M, Simon D I. Platelet expression profiling and clinical validation of myeloid-related protein-14 as a novel determinant of cardiovascular events. Circulation. 2006; 113:2278-2284.
53. Chung I, Lip G Y. Platelets and heart failure. Eur Heart J. 2006; 27:2623-2631.
143. Chung I, Lip G Y. Antithrombotic therapy for congestive heart failure. Int Clin Pract. 2006; 60:36-47.
54. Mackman et al. Complete sequence of the human tissue factor gene, a highly regulated cellular receptor that initiates the coagulation protease cascade. Biochemistry 1989; 28:1755-1762.
55. Del Conde, I., C. N. Shrimpton, P. Thiagarajan, and J. A. Lopez. Tissue-factor-bearing microvesicles arise from lipid rafts and fuse with activated platelets to initiate coagulation. Blood. 2005; 106:1604-1611.
56. Weyrich A S, Elstad M R, McEver R P, McIntyre T M, Moore K L, Morrissey J H, Prescott S M, Zimmerman G A. Activated platelets signal chemokine synthesis by human monocytes. J Clin Invest. 1996; 97:1525-1534.
57. Levy M M, Fink M P, Marshall J C, Abraham E, Angus D, Cook D, Cohen J, Opal S M, Vincent J L, Ramsay G. 2001 SCCM/ESICM/ACCP/ATS/SIS International Sepsis Definitions Conference. Crit. Care Med. 2003; 31:1250-1256.
58. Blann A, Lip, G Y H. Platelet involvement in venous thrombosis and pulmonary embolism. In: Gresele P, Page, C P, Fuster, V, Vermylen, J, ed. Platelets in Thrombotic and Non-thrombotic disorders. Cambridge: Cambridge University Press; 2002:753-760.
59. Geerts W H, Pineo G F, Heit J A, Bergqvist D, Lassen M R, Colwell C W, Ray J G. Prevention of venous thromboembolism: the Seventh ACCP Conference on Antithrombotic and Thrombolytic Therapy. Chest. 2004; 126:338 S-400S.
60. Bunescu A, Widman J, Lenkei R, Menyes P, Levin K, Egberg N. Increases in circulating levels of monocyte-platelet and neutrophil-platelet complexes following hip arthroplasty. Clin Sci (Loud). 2002; 102:279-286.
61. Yang L C, Wang C J, Lee T H, Lin F C, Yang B Y, Lin C R, Lee T C. Early diagnosis of deep vein thrombosis in female patients who undergo total knee arthroplasty with measurement of P-selectin activation. J Vasc Surg. 2002; 35:707-712.
62. Schellong S M. Complete compression ultrasound for the diagnosis of venous thromboembolism. Curr Opin Pulm Med. 2004; 10:350-355.
63. Zierler B K. Ultrasonography and diagnosis of venous thromboembolism. Circulation. 2004; 109:I9-14.
64. Schindler O S, Dalziel R. Post-thrombotic syndrome after total hip or knee arthroplasty: incidence in patients with asymptomatic deep venous thrombosis. J Orthop Surg (Hong Kong). 2005; 13:113-119.
65. Carrigan S D, Scott G, and Tabrizian M. Toward Resolving the Challenges of Sepsis Diagnosis. Clinical Chemistry 2004; 50(8):1301-1314

1. A method for providing a diagnosis, prognosis, or prediction of a disorder or disease, the method comprising:
obtaining a blood sample from a subject;
isolating platelet cells from the blood sample;
assaying pre-mRNA splicing in the platelet cells; and
correlating the presence or absence of mRNA splicing with a diagnosis, prognosis, or prediction of a disorder, disease or condition in the subject.

2. The method according to claim 1, further comprising preparing an RNA sample from the isolated platelet cells, and amplifying the RNA Sample.

3. The method according to claim 1, comprising conducting a polymerase chain reaction (PCR) and quantifying a resulting PCR product.

4. The method according to claim 1, wherein assaying pre-mRNA splicing comprises assaying splicing in tissue factor (TF) mRNA or IL-1β mRNA.

5. The method according to claim 1, wherein assaying pre-mRNA splicing comprises assaying for the presence or absence of an intron.

6. The method according to claim 4, further comprising using PCR with at least two primers targeting sequential exons in the TF mRNA.

7. The method according to claim 1, comprising conducting in situ PCR.

8. The method according to claim 1, comprising using an intronic probe to assay for TF pre-mRNA splicing.

9. The method according to claim 1, comprising assaying TF pre-mRNA splicing using reverse transcriptase PCR.

10. The method according to claim 5, comprising assaying for the presence or absence of an intron using PCR with primers targeting exon 4 and exon 5 of the TF mRNA.

11. The method according to claim 10, comprising using SEQ ID NO: 1 and SEQ ID NO: 2 as primers.

12. The method according to claim 5, comprising assaying for the presence or absence of an intron using PCR with primers targeting exon 1 and exon 6 of the TF mRNA.

13. The method according to claim 12, comprising using SEQ ID NO: 3 and SEQ ID NO: 4 as primers.

14. The method according to claim 1, wherein correlating the presence or absence of tissue factor pre-mRNA splicing with a diagnosis, prognosis, or prediction of a disorder, disease or condition in the subject comprises predicting sepsis.

15. The method according to claim 1, further comprising predicting a probability of clinical SIRS or sepsis developing in the subject.

16. The method according to claim 1, comprising measuring TF-dependent coagulation activity in the isolated platelet cells.

17. The method according to claim 1, comprising enriching the sample for a Clk1 kinase and assaying Clk1 activity in the sample.

18. The method according to claim 17, wherein Clk1 activity is assayed using an immune complex kinase assay.

19. The method according to claim 17, wherein the disorder or disease is sepsis and the diagnosis, prognosis, or prediction is for mortality.

20. The method according to claim 1, comprising obtaining a blood sample from an elderly subject and predicting the subject's risk for developing venous thromboembolism.

21.-47. (canceled)


SEQUENCE LISTING (8 sequences)

SEQ ID NO: 1 - DNA, 21 bp, Artificial
  TF pre-mRNA exon four primer
ctcggacagccaacaattcag

SEQ ID NO: 2 - DNA, 20 bp, Artificial
  TF pre-mRNA exon five primer
cgggctgtctgtactcttcc

SEQ ID NO: 3 - DNA, 21 bp, Artificial
  TF pre-mRNA exon 1 primer
ccaactggtagacatggagac

SEQ ID NO: 4 - DNA, 22 bp, Artificial
  TF pre-mRNA exon six primer
cagtagctccaacagtgcttcc

SEQ ID NO: 5 - DNA, 20 bp, Artificial
  in situ primer for intron four
acccatttcttccccaattc

SEQ ID NO: 6 - DNA, 20 bp, Artificial
  in situ primer for intron four
gtgcctgggatcctcaatag

SEQ ID NO: 7 - DNA, 22 bp, Artificial
  In situ exon 3 primer
ctccccagagttcacaccttac

SEQ ID NO: 8 - DNA, 20 bp, Artificial
  second exon five primer
cgggctgtctgtactcttcc